(19) United States
(12) Patent Application Publication
(10) Pub. No.: US 2018/0255721 A1
(43) Pub. Date: Sep. 13, 2018

(54) CROP PRODUCT DEVELOPMENT AND SEED TREATMENTS

(71) Applicants: PIONEER HI-BRED INTERNATIONAL, INC., JOHNSTON, IA (US); E. I. DU PONT DE NEMOURS AND COMPANY, WILMINGTON, DE (US)

(72) Inventors: KERI CARSTENS, BOONE, IA (US); ALEX COCHRAN, INDIANAPOLIS, IN (US); JASON HAEGELE, MAHOMET, IL (US); STEVEN MITCHELL, ANKENY, IA (US); KEITH A. O'BRYAN, JOHNSTON, IA (US); STEVEN PASZKIEWICZ, JOHNSTON, IA (US); MARK REISINGER, JOHNSTON, IA (US); MARCO TOAPANTA, PARKVILLE, MO (US); ANDRE TREPANIER, JOHNSTON, IA (US)

(73) Assignees: PIONEER HI-BRED INTERNATIONAL, INC., JOHNSTON, IA (US); E. I. DU PONT DE NEMOURS AND COMPANY, WILMINGTON, DE (US)

(21) Appl. No.: 15/754,452

(22) PCT Filed: Aug. 23, 2016
(86) PCT No.: PCT/US16/48231

§ 371 Date: Feb. 22, 2018

Related U.S. Application Data
(60) Provisional application No. 62/209,318, filed on Aug. 24, 2015.

(51) Int. Cl. A01H 1/04; C12N 15/82; A01N 59/20; A01N 43/653; A01N 63/00; A01N 25/26; A01N 43/40; A01H 1/06

(52) U.S. Cl. CPC A01H 1/04; C12N 15/8286; A01N 59/20; A01N 43/653; C12N 15/8285; A01N 25/26; A01N 43/40; A01H 1/06; A01N 63/00

(57) ABSTRACT

Seed treatments to enhance breeding methods are provided. Methods to increase yield by selectively applying seed treatments to breeding parental lines are provided. Variety responses to seed treatments are useful to provide an integrated, seed treatment, genetics, and traits combination to growers, chosen for a particular geographic location.

### FIELD

The field relates to application of seed treatments, plant breeding and crop product development.

### BACKGROUND

The control of insect pests and plant diseases caused by plant pathogens is important in achieving high crop efficiency. Disease and insect damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

Plant varieties developed to perform better in the presence of seed treatments are useful for breeding purposes and therefore desirable for increasing germplasm diversity for plant breeding.

### SUMMARY

Methods and compositions for improved breeding and variety development through selected use of seed treatment products are disclosed.

A method of increasing crop yield under pest or disease pressure, the method includes:

a. providing a first crop plant that is adapted to grow in a first crop growing environment in the absence of a substantial pest or disease pressure and wherein the first crop plant does not exhibit substantial resistance to one or more pests or diseases;

b. providing a second crop plant that is adapted to grow in a second crop growing environment in the presence of a substantial pest or disease pressure and wherein the second crop plant exhibits substantial resistance to one or more pests or disease but yields less compared to the first crop plant in the absence of said pest or diseases pressure;

c. crossing the first and the second crop plant;

d. obtaining a plurality of seeds from the cross;

e. growing the plurality of seeds treated with one or more seed treatments that enhance resistance to one or more pests or diseases present in the second crop growing environment; and

f. selecting a progeny by evaluating the pest or disease resistance performance in the second crop growing environment in the presence of the substantial pest or disease pressure, thereby increasing the crop yield by growing the progeny or a populations of plants derived from the progeny.

A method of breeding a population of plants includes:

a. providing a first population of seeds treated with a seed applied component, wherein the first population of seeds exhibit a higher yield potential in the presence of a significant disease or pest pressure compared to the first population of plants not treated with the seed applied component;

b. crossing one or more members of the first population of plants with one or more members of a second population of plants to produce a breeding population of plants, wherein the second population of plants is genetically dissimilar to the one or more members of the first population of plants;

c. growing the one or more progeny in a plant growing environment in the presence of a substantial pest or disease pressure, wherein the one or more progeny from the breeding population of plants is treated with the seed applied component; and

d. selecting the one or more progeny and creating or generating one or more breeding parents.

In an embodiment, crop plants are selected from the group consisting of soybean, maize, rice, wheat and canola. In an embodiment, a disease is soybean sudden death syndrome. In an embodiment, pest is selected from the group consisting of wireworm, white grubs, black cutworms, seedcorn maggot, corn root worm, fall armyworm, flea beetle and cutworms, and a combination thereof.

A method of developing an integrated seed product comprising a seed applied component includes:

a. growing a population of seeds treated with a seed applied component, wherein the population of seeds exhibit genetic variability with respect to one or more agronomic traits and wherein the seed applied component is selected to improve the one or more agronomic traits;

b. selecting one or more plants that exhibit increased agronomic performance in the presence of the seed applied component for further breeding; and

c. developing an integrated seed product comprising the seed applied component, wherein the seed applied component enhances the performance of the one or more agronomic traits.

A method of increasing crop yield under pest or disease pressure includes:

a. crossing a first crop plant that is adapted to grow in a first crop growing environment in the absence of a substantial pest or disease pressure and a second crop plant that is adapted to grow in a second crop growing environment in the absence of a substantial pest or disease pressure, wherein the first crop plant and the second crop plant are genetically non-identical;

b. obtaining a plurality of seeds from the cross;

c. growing the plurality of seeds treated with one or more seed applied products to enhance resistance to one or more pests or diseases present in a second crop growing environment to the plurality of seeds, wherein the second crop growing environment is different than the first crop growing environment in the presence of one or more pest or disease; and

d. selecting a progeny by evaluating the pest or disease resistance performance in the second crop growing environment in the presence of the substantial pest or disease pressure.

A method of increasing crop yield in a crop growing environment includes:

a. growing a population of plants from a plurality of seeds treated with one or more seed applied components in a first crop growing environment, wherein the plurality of seeds resulted from a cross between a first crop plant and a second crop plant, both the first and the second crop plants adapted to grow in a second crop growing environment;

b. selecting one or more progeny plants based on the ability of the progeny plants that exhibit increased yield in the second crop growing environment in the presence of the one or more seed applied components, wherein the second crop growing environment is different than the first crop growing environment in a characteristic selected from the group consisting of pest, disease, germination, plant vigor, standability and plant health, and a combination thereof; and

c. increasing crop yield in the second crop growing environment.

A method of producing progeny crop seeds for field planting includes

a. growing a population of parental plants from a plurality of seeds treated with a seed applied component, wherein the seed applied component or the effective amount of such seed applied component is not generally applied to a plurality of progeny seeds to produce grain; and

b. producing a plurality of progeny seeds, wherein the seed applied component is not applied or applied at a lower amount to the progeny seeds as compared to the amount applied to the parental population.

In an embodiment, the seed applied component on the plurality of seeds for the parental population increases an agronomic characteristic selected from the group consisting of seed germination, yield, plant health, disease and pest resistance. In an embodiment, the seed applied component to the plurality of seeds for the parental population improves an agronomic characteristic selected from the group consisting of seed germination, yield, plant health, disease and pest resistance, and a combination thereof for the progeny plants. In an embodiment, the progeny plants do not contain a significant amount of the seed applied component to the parent plants.

A method of reducing the development of a resistant insect through an integrated refuge includes:
(a) providing a first portion of seeds coated with a first seed applied insecticide, and
(b) providing a second portion of seeds coated with a second seed applied insecticide,
wherein the first and second seed applied insecticides function through distinct modes of action in controlling one or more insects and the first and second portions of seeds are present in the same container for planting in a field.

In an embodiment, the first portion of the seeds contain a trait not present in the second portion of the seeds. In an embodiment, the seeds are maize seeds. In an embodiment, the second portion of seeds comprise about 5% to about 25% of the total number of seeds in the container. In an embodiment, the trait is a transgenic trait. In an embodiment, the transgenic trait is due to the expression of a Bacillus thuringiensis insecticidal protein, a bacterial insecticidal protein, a plant insecticidal protein, a RNA targeting one or more insects, or a combination thereof.

A method of developing a specific population of crop seeds coated with a specific seed applied component for a specific location includes
(a) providing the specific population of crop seeds coated with the specific seed applied component, wherein the specific population of seeds was selected to exhibit a desirable characteristic in the presence of the specific seed applied component and wherein the seed applied component was selected for its performance at the specific location, and
(b) growing the specific population of crop seeds in a crop growing environment.

In an embodiment, the specific population of crop seeds exhibit disease tolerance or insect resistance. In an embodiment, the specific population of crop seeds exhibit enhanced plant vigor. In an embodiment, the seed applied component is applied at a rate lower than the rate normally used for a general population of crop seeds and wherein the yield resulting from the specific population of crop seeds is the same or higher as compared to the general population of crop seeds. In an embodiment, the specific population of crop seeds is soybean. In an embodiment, the specific population of crop seeds is selected from the group consisting of soybean, maize, rice, sorghum, alfalfa, canola, cotton and wheat. In an embodiment, the specific location is chosen based on an environmental factor selected from the group consisting of pest pressure, disease pressure, soil type, temperature, humidity, day length, and a combination thereof.

A method of altering maturity of a plant, the method comprising
(a) providing a variety of crop seeds coated with a seed applied component, wherein the seed applied component is selected to alter the maturity of a variety of crop plants grown from the variety of crop seeds coated with the seed applied component; and
(b) growing the variety of crop seeds coated with the seed applied component in a crop growing environment by planting the crop seeds within a planting window that is not generally associated with said variety of crop plants, wherein the variety of crop plants display altered maturity as compared to a control variety of plants grown from a control crop seeds not treated with the seed applied component.

In an embodiment, the maturity of the crop plants treated with the seed applied component is shortened to increase yield in the crop growing environment compared to the control crop seeds not treated with the seed applied component. In an embodiment, the maturity of the crop plants treated with the seed applied component is increased to increase yield in the crop growing environment compared to the control crop seeds not treated with the seed applied component. In an embodiment, the maturity of the crop plants treated with the seed applied component is altered by about one relative maturity group. In an embodiment, the plant is soybean and the maturity is altered by up to about two relative maturity groups. In an embodiment, the plant is corn and the maturity is altered by up to about 20 CRM. In an embodiment, the plant is rice, wheat, cotton, sorghum, and canola.

### DETAILED DESCRIPTION

The current disclosure provides methods for improved breeding and hybrid/variety development through selective application of seed treatments and adapting plants to a increasing yield and/or improved performance under agronomic stress such as plant pests and drought stress.

A method of increasing crop yield under pest or disease pressure, the method includes providing a first crop plant, such as for example, a soybean plant that is adapted to grow in a first crop growing environment (e.g., such as dry, low disease pressure) in the absence of a substantial pest or disease pressure and wherein the first crop plant does not exhibit substantial resistance to one or more pests or diseases that may be present in another growing environment (e.g., high moisture, poorly drained soil); providing a second crop plant, such as for example, another soybean plant variety, that is adapted to grow in a second crop growing environment (e.g., high moisture, poorly drained soil) in the presence of a substantial pest or disease pressure (e.g., Phytopthora, SDS and SCN for soybean) and wherein the second crop plant exhibits substantial resistance (e.g., native trait tolerance to SDS or SCN or Phytopthora) to one or more pests or disease but yields less compared to the first crop plant in the absence of said pest or diseases pressure; crossing the first and the second crop plant by cross-pollinating from plant variety to another; obtaining a plurality of seeds resulting from the cross or such breeding efforts; growing the plurality of seeds treated with one or more seed treatments that specifically enhance resistance to one or more pests or diseases present in the second crop growing environment; increasing crop yield by selecting for one or more progeny plants that yield higher under the presence of the substantial pest or disease pressure in the second crop growing environment.

Deliberate selection and breeding of plant varieties that are high-yielding in a disease-free or low disease pressure location crop with appropriate seed applied component or seed treatments that target specific pests and diseases present in a different crop growing environment enhances the breeding germplasm availability and furthers germplasm diversity. Instead of being discarded by breeders for lacking tolerance to certain diseases or pests, the targeted breeding of such germplasm with one or more seed treatments, helps advance those germplasm into commercial products, thereby increasing the yield potential of a wider variety of germplasm, even for high pest or disease pressure locations. Further, early-stage breeding efforts with the selective application of one or more seed treatments to parental lines (including inbreds and varieties) increases the likelihood of advancing one or more desirable genotypes, which otherwise might have been discarded because of their tighter linkage or association with one or more undesirable characteristics—e.g., low disease tolerance or pest susceptibility.

Targeted application of specific seed treatments to earlier breeding population plant material is different than mere selecting a particular variety or a commercial hybrid (that have been previously advanced through traditional breeding and selection approaches) and applying a commercially available seed treatment to overcome diseases or pest infestation. That is, unlike combining previously advanced germplasm with commercialized seed treatments, using seed treatment as a breeding tool or factor, relates to the use of seed treatments earlier in a breeding program to diversify and broaden the available germplasm, so that a holistic product concept based on germplasm (genetic makeup), traits, and seed treatment is developed for a specific environment (i.e., climatic and pest/disease pressures). Therefore, the methods and compositions described herein enable development of a total seed-based product solution for growers from an earlier stage versus simply combining existing commercial-stage germplasm/traits with existing commercial-stage seed treatment or seed applied components.

A method of breeding a population of plants includes providing a first population of seeds treated with a seed applied component, wherein the first population of seeds exhibit a higher yield potential (i.e., in the absence of a seed treatment, they yield less) in the presence of a significant disease or pest pressure compared to the first population of plants not treated with the seed applied component; crossing (breeding) one or more members of the first population of plants with one or more members of a second population of plants to produce a breeding population of plants, wherein the second population of plants is genetically dissimilar (e.g., by the presence of one or more QTLs, SNPs, transgenes, allelic diversity, non-isoline) to the one or more members of the first population of plants; growing the one or more progeny in a plant growing environment in the presence of a substantial pest or disease pressure, wherein the one or more progeny from the breeding population of plants is treated with the seed applied component; and selecting the one or more progeny and creating or generating one or more breeding parents, wherein such breeding parents are further used to produce progeny either for further breeding or seed production.

In an embodiment, the genetic dissimilarity may include one or transgenes, SNPs, alleles, traits, or a combination thereof, present in one genetic background versus another genetic background. In an embodiment, genetic dissimilarity includes varieties from different genetic backgrounds, e.g., stiff stalk and non-stiff stalk corn varieties.

In an embodiment, crop plants are selected from the group consisting of soybean, maize, rice, wheat and canola. In an embodiment, a disease is soybean sudden death syndrome. In an embodiment, pest is selected from the group consisting of wireworm, white grubs, black cutworms, seed corn maggot, corn root worm, fall armyworm, flea beetle and cutworms, and a combination thereof.

A method of developing an integrated, holistic, completed seed product solution to growers, comprising a seed applied component that includes growing a population of seeds treated with a seed applied component, wherein the population of seeds exhibit genetic variability with respect to one or more agronomic traits and wherein the seed applied component is selected to improve the one or more agronomic traits; selecting from the population of seeds one or more plants that exhibit increased agronomic performance in the presence of the seed applied component for further breeding; and developing an integrated seed product comprising the seed applied component, wherein the seed applied component enhances the performance of the one or more agronomic traits. The seed treatment is used to identify potential interactions with the genetic variability of the plants that are manifested in a variation of the agronomic characteristics of the plants. For example, genetic variability to cold emergence, when coupled with a seed treatment that aids in cold emergence, helps advance plant varieties that may be susceptible to cold emergence on their own, but offer higher yield potential in the presence of a suitable seed treatment.

In an embodiment, the seed contains a genetic marker selected for enhanced agronomic performance in the presence of a seed treatment. In an embodiment, the seed contains a transgene suitable for enhanced agronomic performance in the presence of a seed treatment. In an embodiment, the seed contains one or more SNPs selected for increased agronomic performance in the presence of a seed treatment. Agronomic performance may include characteristics such as disease tolerance, pest resistance, drought tolerance, and cold tolerance.

A method of increasing crop yield under pest or disease pressure includes crossing/breeding a first crop plant that is adapted to grow (e.g., that was bred or developed under certain environmental conditions or screens) in a first crop growing environment in the absence of a substantial pest or disease pressure and a second crop plant that is adapted to grow in a second crop growing environment in the absence of a substantial pest or disease pressure, wherein the first crop plant and the second crop plant are genetically non-identical and the first and the second crop plants were developed in the absence of any seed treatment targeting the one or more pests present in the second crop growing environment; obtaining a plurality of seeds from the resulting cross; growing the plurality of seeds treated with one or more seed applied products to enhance resistance to one or more pests or diseases present in a second crop growing environment to the plurality of seeds, wherein the second crop growing environment is different than the first crop growing environment in the presence of one or more pest or disease; and selecting a progeny by evaluating the pest or disease resistance performance in the second crop growing environment in the presence of the substantial pest or disease pressure.

In an embodiment, the first crop plant has been bred for increased yield in the first crop growing environment, in the absence of pest/disease pressure and in the absence of a seed applied component to target the pest/disease. In an embodiment, the second crop plant has been bred for increased yield in the second crop growing environment, in the absence of pest/disease pressure and in the absence of a seed applied component to target the pest/disease. Crossing such first and second crop plants are expected to produce progeny plants that generally do not display resistance or tolerance to a crop growing environment with increased pest or disease pressure. However, in the presence of a seed applied component (such as an insecticide or a fungicide), progeny plants with a different genetic background are adapted to grow in a pest/disease pressure region in the presence of a seed applied component.

A method of increasing soybean yield in a soybean growing environment includes growing a population of soybean plants from a plurality of seeds treated with one or more seed applied components in a first soybean growing environment, wherein the plurality of seeds was obtained from a resulting cross between a first soybean plant and a second soybean plant, both the first and the second soybean plants adapted to grow in a second soybean growing environment (the first and second soybean growing environments are different, e.g., different maturity zones, or different disease/pest pressures); selecting one or more progeny soybean plants based on the ability of the progeny soybean plants that exhibit increased yield in the second soybean growing environment in the presence of the one or more seed applied components. In an embodiment, the second soybean growing environment is different than the first soybean growing environment in a characteristic selected from the group consisting of pest, disease, germination, plant vigor, standability and plant health, and a combination thereof; and increasing soybean yield in the second crop growing environment.

A method of producing progeny crop seeds for field planting includes growing a population of parental plants (e.g., parental inbreds or varieties) from a plurality of seeds treated with a seed applied component, wherein the seed applied component or the effective amount (e.g., higher than normal dosage than what may be applied to target a lower pest pressure) of such seed applied component is not generally applied to a plurality of progeny seeds to produce grain; and producing a plurality of progeny seeds, wherein the seed applied component is not applied or applied at a lower amount to the progeny seeds as compared to the amount applied to the parental population.

In an embodiment, the seed treatment applied to the parent seeds impart epigenetic changes in the progeny seeds and/or plants so that the same treatment is not needed or needed at a reduced level to achieve performance against a particular disease/pest or improved agronomics. In an embodiment, the seed treatment applied at a higher dose may result in a lower seed yield, that is within the tolerance for seed production fields but may not be desirable at a grower field, while the resulting benefits are realized during grain production even if the seed applied component is not applied to the seeds that are planted at the grower location.

In an embodiment, the seed applied component on the plurality of seeds for the parental population increases an agronomic characteristic selected from the group consisting of seed germination, yield, plant health, disease and pest resistance. In an embodiment, the seed applied component to the plurality of seeds for the parental population improves an agronomic characteristic selected from the group consisting of seed germination, yield, plant health, disease and pest resistance for the progeny plants. In an embodiment, the progeny plants do not contain a significant amount of the seed applied component to the parent plants.

A method of reducing the development of a resistant insect through an integrated refuge includes providing a first portion of seeds coated with a first seed applied insecticide, and providing a second portion of seeds coated with a second seed applied insecticide, wherein the first and second seed applied insecticides function through distinct modes of action in controlling one or more insects and the first and second portions of seeds are present in the same container (e.g., a bag, or a bulk storage medium) for planting in a field. In an embodiment, the first seed applied insecticide is a neonicotinoid and the second seed applied component is a non-neonicotinoid (e.g., chlorantraniliprole, cyantraniliprole). In an embodiment, the refuge seeds may also contain fungicides with different mode of actions compared to the fungicides present in the non-refuge seeds in the same container. For example, one class of fungicide present in the refuge seeds is not a SDHI (succinate dehydrogenase inhibitor), whereas the refuge seeds contain one or more SDHI (e.g., fluopyram and penthiopyrad). In an embodiment, the refuge seeds are not part of the same container, that is, the refuge is not integrated within the same container such as a bag.

In an embodiment, seeds are corn seeds. In an embodiment, the seeds are cotton seeds. In an embodiment, the seeds are soybean seeds. In an embodiment, the first portion of the seeds contain a trait not present in the second portion of the seeds. In an embodiment, the seeds are maize seeds. In an embodiment, the second portion of seeds comprise about 5% to about 25% of the total number of seeds in the container. In an embodiment, the trait is a transgenic trait. In an embodiment, the transgenic trait is due to the expression of a Bacillus thuringiensis insecticidal protein or a RNA targeting one or more insects.

A method of developing a specific population of crop seeds coated with a specific seed applied component for a specific location includes providing the specific population of crop seeds coated with the specific seed applied component, wherein the specific population of seeds was selected to exhibit a desirable characteristic in the presence of the specific seed applied component and wherein the seed applied component was selected for its performance at the specific location, and growing the specific population of crop seeds in a crop growing environment.

In an embodiment, the crop seeds are soybean seeds and the specific desirable characteristic is tolerance or resistance to SDS and/or SCN. In an embodiment, the specific population of crop seeds exhibit disease tolerance or insect resistance. In an embodiment, the specific population of crop seeds exhibit enhanced plant vigor. In an embodiment, the seed applied component is applied at a rate lower than the rate normally used for a general population of crop seeds and wherein the yield resulting from the specific population of crop seeds is the same or higher as compared to the general population of crop seeds. In an embodiment, the specific population of crop seeds is soybean. In an embodiment, the specific population of crop seeds is selected from the group consisting of soybean, maize, rice, sorghum, alfalfa, canola, cotton and wheat. In an embodiment, the specific location is chosen based on an environmental factor selected from the group consisting of pest pressure, disease pressure, soil type, temperature, humidity and day length.

In traditional breeding, use of a hybrid (recurrent) parent or parents for recurrent crossing provides a progeny population, where specific genes (e.g., transgenes, loci) and alleles are introduced and maintained through e.g., phenotypic evaluation or by linkage markers for identification and selection (marker-assisted selection) during recurrent crossing. Similar to the breeding of transgenes or alleles, use of a seed treatment on the breeding pair and carrying it through the breeding development can be considered as “forward breeding”, as if seed treatment is considered as a trait. Thus, the effect of seed treatment on the newly developed/crossed selection populations help produce unique genetic and seed treatment combinations, where the performance of the inbred or the resulting hybrid is evaluated in the presence of the seed treatment.

The methods described herein for creating and maintaining a genetically-diverse selection population for use as donor cultivars for self-fertilizing species (e.g., soybeans) and inbred line parents for commercial hybrids (e.g. corn) of either self- or cross-fertilizing plant species with the help of seed treatments.

Recurrent back-crossing to one or more hybrid parents is an effective method for “forward breeding”, where new inbred lines with one or more chosen traits such as transgenes. In some of the embodiments herein, such forward breeding is performed with seed treatments. Use of this method where one or more seed treatments is used as a trait to be selected for, facilitates developing unique, new inbred parents. Following each backcross this process can proceed by self- or sib-mating each individual plant with or without prior selection for one or more traits using either phenotypic (e.g., disease or pest pressure) or genotypic (e.g., marker-assisted) selection, where variety or hybrid by seed treatment interactions are carried forward.

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein, the term “allele” refers to a variant or an alternative sequence form at a genetic locus. In diploids, single alleles are inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the phrase “associated with” refers to a recognizable and/or assayable relationship between two entities. For example, the phrase “associated with a trait” refers to a locus, gene, allele, marker, phenotype, etc., or the expression thereof, the presence or absence of which can influence an extent, degree, and/or rate at which the trait is expressed in an individual or a plurality of individuals.

As used herein, the term “backcross”, and grammatical variants thereof, refers to a process in which a breeder crosses a progeny individual back to one of its parents: for example, a first generation F₁ with one of the parental genotypes of the F₁ individual.

As used herein, the phrase “breeding population” refers to a collection of individuals from which potential breeding individuals and pairs are selected. A breeding population can be a segregating population.

A “candidate set” is a set of individuals that are genotyped at marker loci used for genomic prediction. The candidates may be hybrids.

As used herein, the term “chromosome” is used in its art-recognized meaning as a self-replicating genetic structure containing genomic DNA and bearing in its nucleotide sequence a linear array of genes.

As used herein, the terms “cultivar” and “variety” refer to a group of similar plants that by structural and/or genetic features and/or performance can be distinguished from other members of the same species.

As used herein, “crop growing environment” generally refers to one or more environmental considerations such as soil moisture, temperature, humidity, pest or disease pressure, day length, soil type, soil nutrient, and any other environmental factor that has a material impact on the germination and growth of crop plants such as corn, soybean, canola, rice, wheat, cotton, sorghum, barley and others.

The term “effective amount” as used herein as it relates to crop yield or crop vigor refers to an amount of compound effective to increase crop yield or crop vigor.

“Crop yield” as defined herein refers to the return of crop material obtained after harvesting a plant crop. An increase in crop yield refers to an increase in crop yield relative to an untreated control crop.

“Crop vigor” refers to rate of growth or biomass accumulation of a crop plant. An “increase in vigor refers” to an increase in growth or biomass accumulation in crop plants relative to an untreated control crop.

As used herein, the phrase “determining the genotype” or “analyzing genotypic variation” or “genotypic analysis” of an individual refers to determining at least a portion of the genetic makeup of an individual and particularly can refer to determining genetic variability in an individual that can be used as an indicator or predictor of a corresponding phenotype. Determining a genotype can comprise determining one or more haplotypes or determining one or more polymorphisms exhibiting linkage disequilibrium to at least one polymorphism or haplotype having genotypic value.

Determining the genotype of an individual can also comprise identifying at least one polymorphism of at least one gene and/or at one locus; identifying at least one haplotype of at least one gene and/or at least one locus; or identifying at least one polymorphism unique to at least one haplotype of at least one gene and/or at least one locus. Genotypic variations may also include inserted transgenes or other changes engineered in the host genome.

A “doubled haploid plant” is a plant that is developed by the doubling of a haploid set of chromosomes. A doubled haploid plant is homozygous.

As used herein, the phrase “elite line” refers to any line that is substantially homozygous and has resulted from breeding and selection for superior agronomic performance.

As used herein, the term “gene” refers to a hereditary unit including a sequence of DNA that occupies a specific location on a chromosome and that contains genetic instructions for a particular characteristic or trait in an organism.

As used herein, the phrase “genetic gain” refers to an amount of an increase in performance that is achieved through artificial genetic improvement programs. The term “genetic gain” can refer to an increase in performance that is achieved after one generation has passed.

As used herein, the phrase “genetic map” refers to an ordered listing of loci usually related to the relative positions of the loci on a particular chromosome.

As used herein, the phrase “genetic marker” refers to a nucleic acid sequence (e.g., a polymorphic nucleic acid sequence) that has been identified as being associated with a trait, locus, and/or allele of interest and that is indicative of and/or that can be employed to ascertain the presence or absence of the trait, locus, and/or allele of interest in a cell or organism. Examples of genetic markers include, but are not limited to genes, DNA or RNA-derived sequences (e.g., chromosomal subsequences that are specific for particular sites on a given chromosome), promoters, any untranslated regions of a gene, microRNAs, short inhibitory RNAs (siRNAs; also called small inhibitory RNAs), quantitative trait loci (QTLs), transgenes, mRNAs, double-stranded RNAs, transcriptional profiles, and methylation patterns.

As used herein, the phrase “genetic variability” generally refers to one or more genetic variations in a plant's germplasm that includes quantitative trait loci, SNPs, transgenes and other allelic variations that contribute to one or more observable agronomic phenotypes such as yield, disease resistance, drought, nutrient uptake, insect resistance, and other abiotic & biotic stress tolerance.

As used herein, the term “genotype” refers to the genetic makeup of an organism. Expression of a genotype can give rise to an organism's phenotype (i.e., an organism's observable traits). A subject's genotype, when compared to a reference genotype or the genotype of one or more other subjects, can provide valuable information related to current or predictive phenotypes. The term “genotype” thus refers to the genetic component of a phenotype of interest, a plurality of phenotypes of interest, and/or an entire cell or organism.

As used herein, “haplotype” refers to the collective characteristic or characteristics of a number of closely linked loci within a particular gene or group of genes, which can be inherited as a unit. For example, in some embodiments, a haplotype can comprise a group of closely related polymorphisms (e.g., single nucleotide polymorphisms; SNPs). A haplotype can also be a characterization of a plurality of loci on a single chromosome (or a region thereof) of a pair of homologous chromosomes, wherein the characterization is indicative of what loci and/or alleles are present on the single chromosome (or the region thereof).

As used herein, the term “heterozygous” refers to a genetic condition that exists in a cell or an organism when different alleles reside at corresponding loci on homologous chromosomes.

As used herein, the term “homozygous” refers to a genetic condition existing when identical alleles reside at corresponding loci on homologous chromosomes. It is noted that both of these terms can refer to single nucleotide positions, multiple nucleotide positions (whether contiguous or not), and/or entire loci on homologous chromosomes.

As used herein, the term “hybrid”, when used in the context of a plant, refers to a seed and the plant the seed develops into that results from crossing at least two genetically different plant parents.

As used herein, the term “inbred” refers to a substantially or completely homozygous individual or line. It is noted that the term can refer to individuals or lines that are substantially or completely homozygous throughout their entire genomes or that are substantially or completely homozygous with respect to subsequences of their genomes that are of particular interest.

As used herein, the term “introgress”, and grammatical variants thereof (including, but not limited to “introgression”, “introgressed”, and “introgressing”), refer to both natural and artificial processes whereby one or more genomic regions of one individual are moved into the genome of another individual to create germplasm that has a new combination of genetic loci, haplotypes, and/or alleles. Methods for introgressing a trait of interest can include, but are not limited to, breeding an individual that has the trait of interest to an individual that does not and backcrossing an individual that has the trait of interest to a recurrent parent.

As used herein, “linkage disequilibrium” (LD) refers to a derived statistical measure of the strength of the association or co-occurrence of two distinct genetic markers. Various statistical methods can be used to summarize LD between two markers but in practice only two, termed D′ and r², are widely used. As such, the phrase “linkage disequilibrium” refers to a change from the expected relative frequency of gamete types in a population of many individuals in a single generation such that two or more loci act as genetically linked loci.

As used herein, the phrase “linkage group” refers to all of the genes or genetic traits that are located on the same chromosome. Within a linkage group, those loci that are sufficiently close together physically can exhibit linkage in genetic crosses. Since the probability of a crossover occurring between two loci increases with the physical distance between the two loci on a chromosome, loci for which the locations are far removed from each other within a linkage group might not exhibit any detectable linkage in direct genetic tests. The term “linkage group” is mostly used to refer to genetic loci that exhibit linked behavior in genetic systems where chromosomal assignments have not yet been made. Thus, in the present context, the term “linkage group” is synonymous with the physical entity of a chromosome, although one of ordinary skill in the art will understand that a linkage group can also be defined as corresponding to a region (i.e., less than the entirety) of a given chromosome.

As used herein, the term “locus” refers to a position on a chromosome of a species, and can encompass a single nucleotide, several nucleotides, or more than several nucleotides in a particular genomic region.

As used herein, the terms “marker” and “molecular marker” are used interchangeably to refer to an identifiable position on a chromosome the inheritance of which can be monitored and/or a reagent that is used in methods for visualizing differences in nucleic acid sequences present at such identifiable positions on chromosomes. A marker can comprise a known or detectable nucleic acid sequence. Examples of markers include, but are not limited to genetic markers, protein composition, peptide levels, protein levels, oil composition, oil levels, carbohydrate composition, carbohydrate levels, fatty acid composition, fatty acid levels, amino acid composition, amino acid levels, biopolymers, starch composition, starch levels, fermentable starch, fermentation yield, fermentation efficiency, energy yield, secondary compounds, metabolites, morphological characteristics, and agronomic characteristics. Molecular markers include, but are not limited to restriction fragment length polymorphisms (RFLPs), random amplified polymorphic DNA (RAPD), amplified fragment length polymorphisms (AFLPs), single strand conformation polymorphism (SSCPs), single nucleotide polymorphisms (SNPs), insertion/deletion mutations (indels), simple sequence repeats (SSRs), microsatellite repeats, sequence-characterized amplified regions (SCARs), cleaved amplified polymorphic sequence (CAPS) markers, and isozyme markers, microarray-based technologies. Assay markers, nucleic acid sequences, or combinations of the markers described herein, which can be employed to define a specific genetic and/or chromosomal location.

A marker may correspond to an amplification product generated by amplifying a nucleic acid with one or more oligonucleotides, for example, by the polymerase chain reaction (PCR). As used herein, the phrase “corresponds to an amplification product” in the context of a marker refers to a marker that has a nucleotide sequence that is the same as or the reverse complement of (allowing for mutations introduced by the amplification reaction itself and/or naturally occurring and/or artificial alleleic differences) an amplification product that is generated by amplifying a nucleic acid with a particular set of oligonucleotides. In some embodiments, the amplifying is by PCR, and the oligonucleotides are PCR primers that are designed to hybridize to opposite strands of a genomic DNA molecule in order to amplify a genomic DNA sequence present between the sequences to which the PCR primers hybridize in the genomic DNA. The amplified fragment that results from one or more rounds of amplification using such an arrangement of primers is a double stranded nucleic acid, one strand of which has a nucleotide sequence that comprises, in 5′ to 3′ order, the sequence of one of the primers, the sequence of the genomic DNA located between the primers, and the reverse-complement of the second primer. Typically, the “forward” primer is assigned to be the primer that has the same sequence as a subsequence of the (arbitrarily assigned) “top” strand of a double-stranded nucleic acid to be amplified, such that the “top” strand of the amplified fragment includes a nucleotide sequence that is, in 5′ to 3′ direction, equal to the sequence of the forward primer—the sequence located between the forward and reverse primers of the top strand of the genomic fragment—the reverse-complement of the reverse primer. Accordingly, a marker that “corresponds to” an amplified fragment is a marker that has the same sequence of one of the strands of the amplified fragment.

The term “maturity” as it relates to plant grown, generally refers to that point in time at the end of the grain filling period when maximum weight per kernel has occurred. The usual term for this is “physiological maturity” and is often associated with the development of the black layer at the tip of the mature kernel.

The term “phenotype” refers to any observable property of an organism, produced by the interaction of the genotype of the organism and the environment. A phenotype can encompass variable expressivity and penetrance of the phenotype. Exemplary phenotypes include but are not limited to a visible phenotype, a physiological phenotype, a susceptibility phenotype, a cellular phenotype, a molecular phenotype, and combinations thereof.

As used herein, the term “plant” refers to an entire plant, its organs (i.e., leaves, stems, roots, flowers etc.), seeds, plant cells, and progeny of the same. The term “plant cell” includes without limitation cells within seeds, suspension cultures, embryos, meristematic regions, callus tissue, leaves, shoots, gametophytes, sporophytes, pollen, and microspores. The phrase “plant part” refers to a part of a plant, including single cells and cell tissues such as plant cells that are intact in plants, cell clumps, and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as scions, rootstocks, protoplasts, calli, and the like.

As used herein, the term “polymorphism” refers to the presence of one or more variations of a nucleic acid sequence at a locus in a population of one or more individuals. The sequence variation can be a base or bases that are different, inserted, or deleted. Polymorphisms can be, for example, single nucleotide polymorphisms (SNPs), simple sequence repeats (SSRs), and Indels, which are insertions and deletions. Additionally, the variation can be in a transcriptional profile or a methylation pattern. The polymorphic sites of a nucleic acid sequence can be determined by comparing the nucleic acid sequences at one or more loci in two or more germplasm entries. As such, in some embodiments the term “polymorphism” refers to the occurrence of two or more genetically determined alternative variant sequences (i.e., alleles) in a population. A polymorphic marker is the locus at which divergence occurs. Exemplary markers have at least two (or in some embodiments more) alleles, each occurring at a frequency of greater than 1%. A polymorphic locus can be as small as one base pair (e.g., a single nucleotide polymorphism; SNP).

As used herein, the term “population” refers to a genetically heterogeneous collection of plants that in some embodiments share a common genetic derivation.

As used herein, the term “progeny” refers to any plant that results from a natural or assisted breeding of one or more plants. For example, progeny plants can be generated by crossing two plants (including, but not limited to crossing two unrelated plants, backcrossing a plant to a parental plant, intercrossing two plants, etc.), but can also be generated by selfing a plant, creating an inbred (e.g., a double haploid), or other techniques that would be known to one of ordinary skill in the art. As such, a “progeny plant” can be any plant resulting as progeny from a vegetative or sexual reproduction from one or more parent plants or descendants thereof. For instance, a progeny plant can be obtained by cloning or selfing of a parent plant or by crossing two parental plants and include selfings as well as the F₁ or F₂ or still further generations. An F₁ is a first-generation progeny produced from parents at least one of which is used for the first time as donor of a trait, while progeny of second generation (F₂) or subsequent generations (F₃, F₄, and the like) are in some embodiments specimens produced from selfings (including, but not limited to double haploidization), intercrosses, backcrosses, or other crosses of F₁ individuals, F₂ individuals, and the like. An F₁ can thus be (and in some embodiments, is) a hybrid resulting from a cross between two true breeding parents (i.e., parents that are true-breeding are each homozygous for a trait of interest or an allele thereof, and in some embodiments, are inbred), while an F₂ can be (and in some embodiments, is) a progeny resulting from self-pollination of the F₁ hybrids.

As used herein, the phrase “single nucleotide polymorphism”, or “SNP”, refers to a polymorphism that constitutes a single base pair difference between two nucleotide sequences. As used herein, the term “SNP” also refers to differences between two nucleotide sequences that result from simple alterations of one sequence in view of the other that occurs at a single site in the sequence. For example, the term “SNP” is intended to refer not just to sequences that differ in a single nucleotide as a result of a nucleic acid substitution in one as compared to the other, but is also intended to refer to sequences that differ in 1, 2, 3, or more nucleotides as a result of a deletion of 1, 2, 3, or more nucleotides at a single site in one of the sequences as compared to the other. It would be understood that in the case of two sequences that differ from each other only by virtue of a deletion of 1, 2, 3, or more nucleotides at a single site in one of the sequences as compared to the other, this same scenario can be considered an addition of 1, 2, 3, or more nucleotides at a single site in one of the sequences as compared to the other, depending on which of the two sequences is considered the reference sequence. Single site insertions and/or deletions are thus also considered to be encompassed by the term “SNP”.

As used herein, “seed applied component” generally refers to a seed coating material that may include for example, a fungicide or an insecticide or a nematicide or biological component, or a polymer or a combination of such seed coating agents. Generally, a coating that is applied exogenously to a seed to promote one or more desirable characteristics of the seed or the seedling or the plant is considered a seed applied component.

As used herein, the phrase “substantial pest of disease pressure” generally refers to the severity, intensity and/or frequency of the occurrence of a particular pest or disease in a particular location, based on generally accepted rating or scoring system in practice for that particular pest or disease. For example, a commercial seed supplier for soybeans may rate the tolerance or resistance to SDS on a numerical scale that ranges from 4 to 8 (9=resistant), indicating resistance in elite soybean varieties.

As used herein, the terms “trait” and “trait of interest” refer to a phenotype of interest, a gene that contributes to a phenotype of interest, as well as a nucleic acid sequence associated with a gene that contributes to a phenotype of interest. Any trait that would be desirable to screen for or against in subsequent generations can be a trait of interest. Exemplary, non-limiting traits of interest include yield, disease resistance, agronomic traits, abiotic traits, kernel composition (including, but not limited to protein, oil, and/or starch composition), insect resistance, fertility, silage, and morphological traits. In some embodiments, two or more traits of interest are screened for and/or against (either individually or collectively) in progeny individuals.

As used herein, the phrase “yield potential” generally refers to a seed or a plant's ability to yield higher if appropriate growing conditions are available or provided. For example, a plant is capable of yielding higher if the plant is grown in a relatively pest free or disease free location with moderate to high humidity, but does not yield well if the pest or diseases pressure is present in such a growing location.

A propagule (e.g., a seed) can also be coated with a composition comprising a biologically effective amount of a seed applied component. The coatings of the disclosure are capable of effecting a slow release of a desirable compound by diffusion into the seed and surrounding medium. Coatings include dry dusts or powders adhering to the propagule by action of a sticking agent such as methylcellulose or gum arabic. Coatings can also be prepared from suspension concentrates, water-dispersible powders or emulsions that are suspended in water, sprayed on the propagule in a tumbling device and then dried. Formula I compounds that are dissolved in the solvent can be sprayed on the tumbling propagule and the solvent then evaporated. Such compositions preferably include ingredients promoting adhesion of the coating to the propagule. The compositions may also contain surfactants promoting wetting of the propagule. Solvents used must not be phytotoxic to the propagule; generally water is used, but other volatile solvents with low phytotoxicity such as methanol, ethanol, methyl acetate, ethyl acetate, acetone, etc. may be employed alone or in combination. Volatile solvents are those with a normal boiling point less than about 100° C. Drying must be conducted in a way not to injure the propagule or induce premature germination or sprouting.

Neonicotinoids act as agonists at the nicotinic acetylcholine receptor in the central nervous system of insects. This causes excitation of the nerves and eventual paralysis, which leads to death. Due to the mode of action of neonicotinoids, there is no cross-resistance to conventional insecticide classes such as carbamates, organophosphates, and pyrethroids. A review of the neonicotinoids is described in Pestology 2003, 27, pp 60-63; Annual Review of Entomology 2003, 48, pp 339-364; and references cited therein.

Neonicotinoids act as acute contact and stomach poisons, combine systemic properties with relatively low application rates, and are relatively nontoxic to vertebrates. There are many compounds in this group including the pyridylmethylamines such as acetamiprid and thiacloprid; nitromethylenes such as nitenpyram and nithiazine; nitroguanidines such as clothianidin, dinotefuran, imidacloprid and thiamethoxam.

There are many known insecticides, acaricides and nematicides as disclosed in The Pesticide Manual 13th Ed. 2003 including those whose mode of action is not yet clearly defined and those which are a single compound class including amidoflumet (S 1955), bifenazate, chlorofenmidine, dieldrin, diofenolan, fenothiocarb, flufenerim (UR-50701), metaldehyde, metaflumizone (BASF-320), methoxychlor; bactericides such as streptomycin; acaricides such as chinomethionat, chlorobenzilate, cyhexatin, dienochlor, etoxazole, fenbutatin oxide, hexythiazox and propargite.

The weight ratios of a desirable compound (e.g., a diamide) in the mixtures, compositions and methods of the present disclosure are typically from 150:1 to 1:200, preferably from 150:1 to 1:50, more preferably from 50:1 to 1:10 and most preferably from 5:1 to 1:5. Of note are mixtures, compositions and methods wherein component (b) is a compound selected from (b1) neonicotinoids and the weight ratio of component (b) to the diamide compound, an N-oxide, or a salt thereof is from 150:1 to 1:200. Also of note are mixtures, compositions and methods wherein component (b) is a compound selected from (b2) cholinesterase inhibitors and the weight ratio of component (b) to the diamide compound, an N-oxide, or a salt thereof is from 200:1 to 1:100. Also of note are mixtures, compositions and methods wherein component (b) is a compound selected from (b3) sodium channel modulators and the weight ratio of component (b) to the diamide, an N-oxide, or a salt thereof is from 100:1 to 1:10.

TABLE A
Exemplary Seed treatment combination list

| Crop/Seed | Combinations of one or more components listed below | | |
|---|---|---|---|
| Treatment | | Fungicide (includes | Other Seed Treatment |
| Combinations | Insecticide | nematicide) | Components |
| Corn | Thiamethoxam, | Azoxystrobin, Fludioxonil, | Bacillus firmus I-1582, |
| | Clothianidin, | Picoxystrobin, Mefenoxam, | Bacillus subtilis, Bacillus |
| | Chlorantraniliprole, | Ipconazole, Thiabendazole, | simplex, Abamectin, |
| | Cyantraniliprole, | Tebuconazol, Prothioconazole | Polymeric Polyhydroxy |
| | Sulfoxaflor, | Penthiopyrad, oxathiapiprolin, | Acids |
| | Thiacloprid, | Fluopyram, Tioxazafen | |
| | Flupyradifurone | | |
| Soybean | Imidacloprid, | Metalaxyl, Trifloxystrobin, | Bradyrhizobium japonicum, |
| | Thiamethoxam, | Penthiopyrad, Ipconazole, | Bacillus firmus I-1582, |
| | Chlorantraniliprole, | Oxathiapiprolin, Sedaxane, | Bacillus subtilis, Bacillus |
| | Cyantraniliprole | Penflufen, Prothioconazole, | simplex, Pasteuria |
| | Sulfoxaflor, | Difenoconazole, Fluopyram, | nishizawae |
| | Thiacloprid, | Tioxazafen | |
| | Flupyradifurone | | |
| Canola | Thiamethoxam, | Metalaxyl, Picoxystrobin, | Penicillium bilaii, |
| | Clothianidin, | Penthiopyrad, Difenoconazole, | |
| | Chlorantraniliprole, | Trifloxystrobin, Penflufen, | |
| | Cyantraniliprole | Fludioxonil | |
| | Sulfoxaflor | | |

### EXAMPLES

The present disclosure is further illustrated in the following Examples. It should be understood that these Examples, while indicating embodiments of the invention, are given by way of illustration only. Thus, various modifications in addition to those shown and described herein will be apparent to those skilled in the art from the foregoing description. Such modifications are also intended to fall within the scope of the appended claims.

### Example 1

### Seed Treatment Enabled Soybean Breeding Methods

Soybean varieties are typically developed for use in seed and grain production. However, soybean varieties also provide a source of breeding material that may be used to develop new soybean varieties. Plant breeding techniques known in the art and used in a soybean plant breeding program include, but are not limited to, recurrent selection, mass selection, bulk selection, backcrossing, pedigree breeding, open pollination breeding, restriction fragment length polymorphism enhanced selection, genetic marker enhanced selection, making double haploids, and transformation. Often combinations of these techniques are used. The development of soybean varieties in a plant breeding program requires, in general, the development and evaluation of homozygous varieties. There are many analytical methods available to evaluate a new variety including the traditional method of observation of phenotypic traits as well as genotypic analysis.

In addition, seed treatments can also be used to mitigate symptoms such as iron chlorosis for soybean varieties. Iron deficiency symptoms generally do not show up on cotyledon (seed leaves) or unifoliate (single leaf) leaves. Initial chlorosis symptoms typically occur on the trifoliate leaves, beginning as early as the first trifoliate stage. Symptoms may increase or decrease in intensity during the season depending on growing conditions. Iron chlorosis, due to low availability of iron in high pH (alkaline) soils, in a soybean field generally occurs in spots and often in a random pattern, depending on chemical and physical soil differences in the field.

Selection of a particular soybean variety is often based on a variety of factors such as, yield, disease tolerance/resistance, resistance to pests, standability, plant vigor and other agronomic performances. For example, a particular variety may be chosen for its ability to resist a particular disease. However, a particular variety may not be chosen because of its lack of adequate resistance to a particular disease despite exhibiting a higher yield potential in the absence of such disease pressure. In an embodiment, a particular soybean variety that does not have adequate resistance to a particular disease is chosen such that with application of a seed treatment, that particular variety becomes a suitable parental material for further breeding or for commercial placement of that variety in a high disease pressure location with the presence of a suitable seed treatment. Soybean sudden death syndrome (SDS) is caused by the soilborne fungus Fusarium solanif. sp. glycines, synonym: Fusarium virguliforme. The first noticeable symptoms of SDS are yellowing and defoliation of upper leaves.

TABLE 1
SDS disease tolerant/susceptible soybean varieties performance in SDS
locations

| | Relative | | | | |
|---|---|---|---|---|---|
| | Difference in | | | | |
| Variety + | Yield | | | | |
| Seed Treatment | (bu/acre) | Standard | DF | t Value | Pr > \|t\| |
| Fungicide response: | 8.2 | 1.2994 | 102 | 6.33 | <.0001 |
| Variety 1 | | | | | |
| Fungicide response: | 6.1 | 1.4558 | 127 | 4.21 | <.0001 |
| Variety 2 | | | | | |
| Fungicide response: | 3.6 | 1.277 | 99.7 | 2.83 | 0.0056 |
| Variety 3 | | | | | |
| Fungicide response: | 4.9 | 1.2784 | 96 | 3.82 | 0.0002 |
| Variety 4 | | | | | |
| Fungicide response: | 4.0 | 1.3781 | 115 | 2.9 | 0.0045 |
| Variety 5 | | | | | |

Relative difference in Yield indicates the relative difference in field yield in comparison to a commercial variety that had been selected to perform for the geographical region, where it is commercially available.

TABLE 2
SDS disease tolerant/susceptible soybean varieties performance in non-
SDS locations

| | Relative | | | | |
|---|---|---|---|---|---|
| | Difference in | | | | |
| Variety + | Yield | | | | |
| Seed Treatment | (bu/acre) | Standard | DF | t Value | Pr > \|t\| |
| Fungicide response: | −1.563 | 0.8737 | 220 | −1.79 | 0.075 |
| Variety 1 | | | | | |
| Fungicide response: | −2.3954 | 0.8148 | 208 | −2.94 | 0.0037 |
| Variety 2 | | | | | |
| Fungicide response: | −1.7763 | 0.7969 | 185 | −2.23 | 0.027 |
| Variety 3 | | | | | |
| Fungicide response: | −0.896 | 0.7793 | 184 | −1.15 | 0.2518 |
| Variety 4 | | | | | |
| Fungicide response: | −2.2853 | 0.7504 | 156 | −3.05 | 0.0027 |
| Variety 5 | | | | | |

Relative difference in Yield indicates the relative difference in field yield in comparison to a commercial variety that had been selected to perform for the geographical region, where it is commercially available.

In this Example for Table 1, the soybean varieties used had the following SDS scores—the higher the number the better the resistance/tolerance: Soybean Variety 1 (SDS Score of 5; resistant); Variety 2 (SDS Score of 4; susceptible); Variety 3 (SDS Score of 4; susceptible); Variety 4 (SDS Score of 6; resistant); Variety 5 (SDS Score of 4; susceptible). Fungicide used in the experiments, whose results are shown in Tables 1 and 2 include commercially available fluopyram seed treatment formulation.

For Tables 3 and 4, FSTR stands for Fungicide Seed Treatment Recipe. FSTR 7, 5, 6 are the same throughout. FSTR7: includes fungicide 1 (low rate) and fungicide 2 seed treatments not included in FSTR5. FSTR 6 is a control seed treatment that includes fungicide 1 (high rate); and FSTR 5 does not include fungicides 1 and 2 that are present in FSTR6 and FSTR7. FSTR5 is present in both FSTR6 and 7.

TABLE 3
(A-D): SDS disease tolerant/susceptible soybean varieties yield
performance in SDS locations, by relative maturity grouping.

| | | | | | Relative | |
|---|---|---|---|---|---|---|
| | | | | | difference in | |
| | | | | | yield | |
| Relative | | | | | compared to | |
| Maturity | | Seed | Mean yield | Standard | control | |
| group | Variety | Treatment | (bu/acre) | error | (bu/acre) | P value |
| Early 30 | A | FSTR 7 | 78.44 | 8.26 | 2.499 | 0.302 |
| Early 30 | A | FSTR 5 | 72.45 | 8.26 | −3.488 | 0.150 |
| Early 30 | B | FSTR 6 | 75.68 | 8.27 | −0.266 | 0.849 |
| Early 30 | B | FSTR 7 | 78.35 | 8.27 | 2.412 | 0.326 |
| Early 30 | B | FSTR 5 | 73.39 | 8.27 | −2.554 | 0.298 |
| Early 30 | C | FSTR 6 | 76.03 | 8.26 | 0.090 | 0.948 |
| Early 30 | C | FSTR 7 | 78.50 | 8.26 | 2.563 | 0.294 |
| Early 30 | C | FSTR 5 | 72.34 | 8.27 | −3.601 | 0.142 |
| Early 30 | D | FSTR 6 | 76.75 | 8.26 | 0.805 | 0.560 |
| Early 30 | D | FSTR 7 | 77.67 | 8.26 | 1.731 | 0.477 |
| Early 30 | D | FSTR 5 | 73.34 | 8.26 | −2.599 | 0.286 |
| Early 30 | E | FSTR 6 | 76.34 | 8.26 | 0.394 | 0.776 |
| Early 30 | E | FSTR 7 | 78.09 | 8.26 | 2.144 | 0.378 |
| Early 30 | E | FSTR 5 | 72.76 | 8.27 | −3.184 | 0.193 |
| Early 30 | F | FSTR 6 | 76.33 | 8.26 | 0.390 | 0.778 |
| Early 30 | F | FSTR 7 | 78.44 | 8.26 | 2.496 | 0.307 |
| Early 30 | F | FSTR 5 | 71.39 | 8.26 | −4.554 | 0.062 |
| Early 30 | B | FSTR 6 | 75.94 | 8.26 | 0.000 | |
| | | CONTROL | | | | |

(B)

| | | | | | | |
|---|---|---|---|---|---|---|
| Mid 30 | D | FSTR 6 | 69.13 | 4.33 | −0.600 | 0.786 |
| Mid 30 | D | FSTR 7 | 70.24 | 4.33 | 0.512 | 0.824 |
| Mid 30 | D | FSTR 5 | 62.91 | 4.33 | −6.821 | 0.003 |
| Mid 30 | E | FSTR 6 | 72.39 | 4.33 | 2.659 | 0.230 |
| Mid 30 | E | FSTR 7 | 73.16 | 4.34 | 3.424 | 0.142 |
| Mid 30 | E | FSTR 5 | 70.04 | 4.33 | 0.306 | 0.894 |
| Mid 30 | F | FSTR 6 | 69.84 | 4.33 | 0.108 | 0.961 |
| Mid 30 | F | FSTR 7 | 71.30 | 4.33 | 1.566 | 0.497 |
| Mid 30 | F | FSTR 5 | 63.07 | 4.33 | −6.662 | 0.004 |
| Mid 30 | G | FSTR 7 | 71.98 | 4.33 | 2.254 | 0.178 |
| Mid 30 | G | FSTR 5 | 66.00 | 4.33 | −3.732 | 0.026 |
| Mid 30 | H | FSTR 6 | 72.27 | 4.33 | 2.537 | 0.252 |
| Mid 30 | H | FSTR 7 | 73.30 | 4.33 | 3.572 | 0.122 |
| Mid 30 | H | FSTR 5 | 67.49 | 4.33 | −2.242 | 0.331 |
| Mid 30 | I | FSTR 6 | 68.85 | 4.34 | −0.878 | 0.693 |
| Mid 30 | I | FSTR 7 | 69.51 | 4.33 | −0.220 | 0.924 |
| Mid 30 | I | FSTR 5 | 62.28 | 4.33 | −7.448 | 0.001 |
| Mid 30 | G | FSTR 6 | 69.73 | 4.33 | 0.000 | |
| | | CONTROL | | | | |

(C)

| | | | | | | |
|---|---|---|---|---|---|---|
| Late 30 | J | FSTR 6 | 66.25 | 6.09 | 2.910 | 0.158 |
| Late 30 | J | FSTR 7 | 67.45 | 6.09 | 4.107 | 0.073 |
| Late 30 | J | FSTR 5 | 63.69 | 6.09 | 0.345 | 0.880 |
| Late 30 | G | FSTR 7 | 64.54 | 6.09 | 1.197 | 0.226 |
| Late 30 | G | FSTR 5 | 60.78 | 6.09 | −2.564 | 0.010 |
| Late 30 | K | FSTR 6 | 63.85 | 6.09 | 0.513 | 0.803 |
| Late 30 | K | FSTR 7 | 65.05 | 6.09 | 1.710 | 0.454 |
| Late 30 | K | FSTR 5 | 61.29 | 6.09 | −2.051 | 0.369 |
| Late 30 | L | FSTR 6 | 64.92 | 6.09 | 1.575 | 0.444 |
| Late 30 | L | FSTR 7 | 66.11 | 6.09 | 2.772 | 0.225 |
| Late 30 | L | FSTR 5 | 62.35 | 6.09 | −0.989 | 0.665 |
| Late 30 | H | FSTR 6 | 64.66 | 6.09 | 1.320 | 0.521 |
| Late 30 | H | FSTR 7 | 65.86 | 6.09 | 2.517 | 0.271 |
| Late 30 | H | FSTR 5 | 62.10 | 6.09 | −1.244 | 0.586 |
| Late 30 | I | FSTR 6 | 61.85 | 6.09 | −1.494 | 0.468 |
| Late 30 | I | FSTR 7 | 63.04 | 6.09 | −0.297 | 0.896 |
| Late 30 | I | FSTR 5 | 59.28 | 6.09 | −4.059 | 0.076 |
| Late 30 | G | FSTR 6 | 63.34 | 6.09 | 0.000 | |
| | | CONTROL | | | | |

(D)

| | | | | | | |
|---|---|---|---|---|---|---|
| Late 40 | M | FSTR 6 | 68.15 | 7.40 | −5.57 | 0.002534 |
| Late 40 | M | FSTR 7 | 66.74 | 7.40 | −6.98 | 0.003317 |
| Late 40 | M | FSTR 5 | 65.56 | 7.40 | −8.17 | 0.000616 |
| Late 40 | N | FSTR 6 | 67.89 | 7.40 | −5.84 | 0.002016 |
| Late 40 | N | FSTR 7 | 66.48 | 7.40 | −7.25 | 0.002731 |
| Late 40 | N | FSTR 5 | 65.30 | 7.40 | −8.43 | 0.000522 |
| Late 40 | O | FSTR 7 | 72.32 | 7.40 | −1.41 | 0.341213 |
| Late 40 | O | FSTR 5 | 71.14 | 7.40 | −2.59 | 0.078908 |
| Late 40 | P | FSTR 6 | 69.19 | 7.40 | −4.54 | 0.01418 |
| Late 40 | P | FSTR 7 | 67.78 | 7.40 | −5.95 | 0.013085 |
| Late 40 | P | FSTR 5 | 66.60 | 7.40 | −7.13 | 0.002733 |
| Late 40 | Q | FSTR 6 | 70.23 | 7.40 | −3.49 | 0.058056 |
| Late 40 | Q | FSTR 7 | 68.82 | 7.40 | −4.90 | 0.03963 |
| Late 40 | Q | FSTR 5 | 67.64 | 7.40 | −6.09 | 0.01046 |
| Late 40 | R | FSTR 6 | 61.68 | 7.40 | −12.05 | 3.35E−10 |
| Late 40 | R | FSTR 7 | 60.27 | 7.40 | −13.45 | 3.58E−08 |
| Late 40 | R | FSTR 5 | 59.09 | 7.40 | −14.64 | 2.15E−09 |
| Late 40 | O | FSTR 6 | 73.73 | 7.40 | 0.00 | |
| | | CONTROL | | | | |

Methods for producing a soybean plant by crossing a first parent soybean plant with a second parent soybean plant wherein the first and/or second parent soybean plant is a variety that may be susceptible to a particular diseases such as SDS. Any such methods include but are not limited to selfing, sibbing, backcrossing, mass selection, pedigree breeding, bulk selection, hybrid production, crossing to populations, and the like. These methods are well known in the art and some of the more commonly used breeding methods are described below. However, a seed treatment specifically chosen to address a particular disease is used in the process of selecting a particular variety for breeding purposes. Seeds from a susceptible variety is treated with one or more seed treatment components and the resulting progeny from a breeding cross are also evaluated in the presence of seed treatment components chosen to address the particular disease—either in the presence of that particular disease or in the absence of such disease pressure.

Thus, varieties that are not traditionally selected for breeding purposes for a particular disease or a specific location are chosen for advancement based on the performance of those varieties or their progenies in the presence of one or more seed treatments or seed applied components. Similarly, varieties that are not adapted to a particular geography or location due to a variety of factors including pest pressure, disease presence, abiotic stresses, climate, soil conditions, and day length can be adapted to grow in the presence of one or more seed treatment components such as insecticides, fungicides, nematicides, plant health components, biologicals and others.

The process of selecting soybean varieties in the presence of seed treatment components enable a breeder to increase the available germplasm pool to improve progeny generation and increase the availability heterotic population groups for breeding purposes.

TABLE 4
(A-D): SDS disease tolerant/susceptible soybean varieties
SDS score, by relative maturity grouping.

| | | | | | Relative | |
|---|---|---|---|---|---|---|
| | | | | | difference | |
| | | | | | in SDS | |
| | | | Mean | | score | |
| Relative | | | SDS | | compared | |
| Maturity | | Seed | score (1-9 | Standard | to control | |
| group | Variety | Treatment | scale) | error | (1-9 scale) | P value |
| Early 30 | A | FSTR 7 | 4.72 | 1.12 | −0.03 | 0.7806 |
| Early 30 | A | FSTR 5 | 4.33 | 1.12 | −0.42 | 0.0006 |
| Early 30 | B | FSTR 6 | 4.75 | 1.12 | 0.00 | 0.9996 |
| Early 30 | B | FSTR 7 | 4.72 | 1.12 | −0.03 | 0.7806 |
| Early 30 | B | FSTR 5 | 4.33 | 1.12 | −0.42 | 0.0006 |
| Early 30 | C | FSTR 6 | 4.75 | 1.12 | 0.00 | 0.9991 |
| Early 30 | C | FSTR 7 | 4.72 | 1.12 | −0.03 | 0.7806 |
| Early 30 | C | FSTR 5 | 4.33 | 1.12 | −0.42 | 0.0006 |
| Early 30 | D | FSTR 6 | 4.75 | 1.12 | 0.00 | 0.9996 |
| Early 30 | D | FSTR 7 | 4.72 | 1.12 | −0.03 | 0.7806 |
| Early 30 | D | FSTR 5 | 4.33 | 1.12 | −0.42 | 0.0006 |
| Early 30 | E | FSTR 6 | 4.75 | 1.12 | 0.00 | 1.0000 |
| Early 30 | E | FSTR 7 | 4.72 | 1.12 | −0.03 | 0.7806 |
| Early 30 | E | FSTR 5 | 4.33 | 1.12 | −0.42 | 0.0006 |
| Early 30 | F | FSTR 6 | 4.75 | 1.12 | 0.00 | 0.9989 |
| Early 30 | F | FSTR 7 | 4.72 | 1.12 | −0.03 | 0.7806 |
| Early 30 | F | FSTR 5 | 4.33 | 1.12 | −0.42 | 0.0006 |
| Early 30 | B | FSTR 6 | 4.75 | 1.12 | 0.00 | |

(B)

| | | | | | | |
|---|---|---|---|---|---|---|
| Mid 30 | D | FSTR 6 | 7.82 | 0.26 | −1.37 | 1.96E−06 |
| Mid 30 | D | FSTR 7 | 7.57 | 0.26 | −1.63 | 4.1E−07 |
| Mid 30 | D | FSTR 5 | 6.19 | 0.26 | −3.01 | 0 |
| Mid 30 | E | FSTR 6 | 8.82 | 0.26 | −0.38 | 0.1741 |
| Mid 30 | E | FSTR 7 | 8.54 | 0.26 | −0.65 | 0.034663 |
| Mid 30 | E | FSTR 5 | 7.16 | 0.26 | −2.04 | 5.89E−10 |
| Mid 30 | F | FSTR 6 | 7.50 | 0.26 | −1.70 | 7.55E−09 |
| Mid 30 | F | FSTR 7 | 7.07 | 0.26 | −2.12 | 1.32E−10 |
| Mid 30 | F | FSTR 5 | 5.54 | 0.26 | −3.66 | 0 |
| Mid 30 | G | FSTR 7 | 9.02 | 0.26 | −0.18 | 0.468877 |
| Mid 30 | G | FSTR 5 | 7.81 | 0.26 | −1.38 | 1.15E−07 |
| Mid 30 | H | FSTR 6 | 7.75 | 0.26 | −1.45 | 5.94E−07 |
| Mid 30 | H | FSTR 7 | 7.52 | 0.26 | −1.68 | 1.85E−07 |
| Mid 30 | H | FSTR 5 | 6.04 | 0.26 | −3.16 | 0 |
| Mid 30 | I | FSTR 6 | 8.26 | 0.26 | −0.94 | 0.000864 |
| Mid 30 | I | FSTR 7 | 8.09 | 0.26 | −1.11 | 0.0004 |
| Mid 30 | I | FSTR 5 | 6.61 | 0.26 | −2.59 | 3.11E−14 |
| Mid 30 | G | FSTR 6 | 9.20 | 0.26 | 0.00 | |
| | | CONTROL | | | | |

(C)

| | | | | | | |
|---|---|---|---|---|---|---|
| Late 30 | J | FSTR 6 | 8.95 | 0.13 | 0.00 | 1 |
| Late 30 | J | FSTR 7 | 9.04 | 0.13 | 0.08 | 0.646 |
| Late 30 | J | FSTR 5 | 8.79 | 0.13 | −0.17 | 0.359 |
| Late 30 | G | FSTR 7 | 9.04 | 0.13 | 0.08 | 0.423 |
| Late 30 | G | FSTR 5 | 8.79 | 0.13 | −0.17 | 0.111 |
| Late 30 | K | FSTR 6 | 8.90 | 0.13 | −0.05 | 0.729 |
| Late 30 | K | FSTR 7 | 8.99 | 0.13 | 0.03 | 0.860 |
| Late 30 | K | FSTR 5 | 8.74 | 0.13 | −0.22 | 0.231 |
| Late 30 | L | FSTR 6 | 8.85 | 0.13 | −0.10 | 0.490 |
| Late 30 | L | FSTR 7 | 8.93 | 0.13 | −0.02 | 0.914 |
| Late 30 | L | FSTR 5 | 8.68 | 0.13 | −0.27 | 0.140 |
| Late 30 | H | FSTR 6 | 8.64 | 0.13 | −0.31 | 0.041 |
| Late 30 | H | FSTR 7 | 8.73 | 0.13 | −0.23 | 0.217 |
| Late 30 | H | FSTR 5 | 8.48 | 0.13 | −0.48 | 0.010 |
| Late 30 | I | FSTR 6 | 8.70 | 0.13 | −0.26 | 0.087 |
| Late 30 | I | FSTR 7 | 8.78 | 0.13 | −0.17 | 0.339 |
| Late 30 | I | FSTR 5 | 8.53 | 0.13 | −0.42 | 0.022 |
| Late 30 | G | FSTR 6 | 8.95 | 0.13 | 0.00 | |
| | | CONTROL | | | | |

(D)

| | | | | | | |
|---|---|---|---|---|---|---|
| Late 40 | M | FSTR 6 | 4.93 | 1.29 | 0.15 | 0.423 |
| Late 40 | M | FSTR 7 | 4.94 | 1.29 | 0.15 | 0.433 |
| Late 40 | M | FSTR 5 | 4.44 | 1.29 | −0.34 | 0.079 |
| Late 40 | N | FSTR 6 | 4.80 | 1.29 | 0.02 | 0.934 |
| Late 40 | N | FSTR 7 | 4.80 | 1.29 | 0.01 | 0.941 |
| Late 40 | N | FSTR 5 | 4.94 | 1.29 | 0.16 | 0.423 |
| Late 40 | O | FSTR 7 | 4.95 | 1.29 | 0.16 | 0.399 |
| Late 40 | O | FSTR 5 | 4.90 | 1.29 | 0.12 | 0.555 |
| Late 40 | P | FSTR 6 | 4.95 | 1.29 | 0.16 | 0.374 |
| Late 40 | P | FSTR 7 | 4.81 | 1.29 | 0.03 | 0.896 |
| Late 40 | P | FSTR 5 | 4.49 | 1.29 | −0.30 | 0.126 |
| Late 40 | Q | FSTR 6 | 4.79 | 1.29 | 0.00 | 0.986 |
| Late 40 | Q | FSTR 7 | 4.95 | 1.29 | 0.16 | 0.417 |
| Late 40 | Q | FSTR 5 | 4.44 | 1.29 | −0.34 | 0.081 |
| Late 40 | R | FSTR 6 | 4.93 | 1.29 | 0.14 | 0.450 |
| Late 40 | R | FSTR 7 | 4.94 | 1.29 | 0.15 | 0.433 |
| Late 40 | R | FSTR 5 | 4.85 | 1.29 | 0.06 | 0.743 |
| Late 40 | O | FSTR 6 | 4.79 | 1.29 | 0.00 | |
| | | CONTROL | | | | |

In an embodiment, pedigree breeding for soybean starts with the crossing of two genotypes, soybean variety A and a soybean variety B having one or more desirable characteristics that is lacking or which complements variety A. If the two original parents do not provide all the desired characteristics, other sources can be included in the breeding population. In the pedigree method, superior plants are selfed and selected in successive filial generations. In the succeeding filial generations, the heterozygous allele condition gives way to the homozygous allele condition as a result of inbreeding or back crossing. After a sufficient amount of inbreeding, successive filial generations will serve to increase seed of the developed variety. Typically, the developed variety comprises homozygous alleles at about 95% or more of its loci. The initial selection of parents from the heterotic groups for breeding purposes depends on a number of characteristics including performance against a particular pest, disease or adaption to a particular environmental condition including day-length. The availability of a particular seed applied component, such as, for example, a fungicide that is effective against SDS in soybean is a factor in selecting breeding pairs and selecting for progeny in the presence of a seed treatment and the presence of SDS.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, and selfed progeny. The selected progeny are cross pollinated with each other to form progeny for another population. This population is planted and, again, superior plants are selected to cross pollinate with each other. Recurrent selection is a cyclical process and therefore can be repeated as many times as desired. The objective of recurrent selection is to improve the traits of a population. The improved population can then be used as a source of breeding material to obtain new varieties for commercial or breeding use, including the production of a synthetic cultivar. A synthetic cultivar is the resultant progeny formed by the intercrossing of several selected varieties. Seed treatments that provide one or more advantages such as for example, disease resistance are used during the breeding program that involves recurrent selection. A specific progeny is selected not for its tolerance to a particular disease (when such protection is provided by one or more seed applied component), but to another trait, such as for example, early vigor or increased yield in the absence of the seed treatment.

Molecular markers, which include markers identified through the use of techniques such as isozyme electrophoresis, restriction fragment length polymorphisms (RFLPs), randomly amplified polymorphic DNAs (RAPDs), arbitrarily primed polymerase chain reaction (AP-PCR), DNA amplification fingerprinting (DAF), sequence characterized amplified regions (SCARs), amplified fragment length polymorphisms (AFLPs), simple sequence repeats (SSRs), and single nucleotide polymorphisms (SNPs), may be used in plant breeding methods seed treatments or seed applied components that specifically address an agronomic characteristic not present in a breeding population or not expressed to a commercially adequate level in the breeding population. These methods are readily applicable to any plant variety that has molecular markers-based breeding methods.

One use of molecular markers is quantitative trait loci (QTL) mapping. QTL mapping is the use of markers which are known to be closely linked to alleles that have measurable effects on a quantitative trait. For example, QTL mapping is a tool to associate a particular variety's response to a particular seed treatment. For example, crop response to a specific seed treatment is analyzed by utilizing one or more molecular markers that are diagnostic of crop response. Selection in the breeding process is based upon the accumulation of markers linked to the positive effecting alleles and/or the elimination of the markers linked to the negative effect alleles from the plant genome.

Molecular markers can also be used during the breeding process for the selection of traits that enhance the plant's positive interaction to a seed applied component. For example, markers closely linked to alleles or markers containing sequences within the actual alleles of interest can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants. It can also be used to reduce the number of crosses back to the recurrent parent needed in a backcrossing program. The use of molecular markers in the selection process is often called genetic marker enhanced selection.

For example, Phytophthora root rot negatively impacts soybean yield, and is caused by Phytophthora megasperma Drechs. In the spring, oospores germinate whenever the temperature is suitable and form sporangia. Sporangia accumulate until the soil is flooded, at which time zoospores are released. Sporangia also form on the surfaces of infected roots, providing secondary inoculum. Zoospores are produced in abundance in flooded and water-logged soils and are disseminated by soil water. Zoospores attract toward the roots, where they encyst and germinate. Hyphae grow intercellularly in root tissues. Leaf infection occurs when soil particles containing the pathogen are deposited on leaves by wind or rainstorms. If the weather remains humid and cloudy, leaves become severely infected, and fungus grows toward the petiole and stem. Phytophthora root rot is most common in heavy, highly compacted, fine-textured (clay) soils subject to flooding. Host resistance is a tool for combating this disease; specifically, race-specific resistance through single dominant rps genes are used, alongside tolerance or partial resistance through multiple genes.

In an embodiment, a particular soybean variety that does not have adequate resistance to a this disease is chosen such that with application of a seed treatment, that particular variety becomes a suitable parental material for further breeding or for commercial placement of that variety in a high disease pressure location with the presence of a suitable seed treatment, (e.g., oxathiapiprolin or metalaxyl). The following data exemplify the response (disease tolerance and yield, respectively) of soybean varieties with different tolerance levels (through multiple genes, scored 1-9) when seed-applied technologies are used, thereby indicating the potential use of parental material or commercial placement of varieties through the use of targeted seed treatment technologies.

For Tables 5A-C, FSTR 1, 2, 3, 4, and 5 are the same throughout, with FSTR 5 does not include the fungicides present in FSTR1-4. FSTR 1 and 3: includes a specific fungicide at a low rate, with the only difference between FSTR 1 and 3 coming from a non-fungicidal component. FSTR 2 and 4: includes the same specific fungicide at a higher rate, with the only difference between FSTR 2 and 4 coming from a non-fungicidal component.

TABLE 5
(A-C): Yield performance of soybean varieties with Phytophthora
susceptibility/tolerance grown in locations with history of Phytophthora disease.

| | | | | | | |
|---|---|---|---|---|---|---|
| | | | | | Relative | |
| | | | | | difference | |
| | | | | | in yield | |
| Relative | | | Mean | | compared | |
| Maturity | | Seed | yield | Standard | to control | |
| group | Variety | Treatment | (bu/acre) | error | (bu/acre) | P value |

(A)

| | | | | | | |
|---|---|---|---|---|---|---|
| Mid 00 | AA | FSTR 1 | 49.69 | 4.69 | −1.55 | 0.212 |
| Mid 00 | AA | FSTR 2 | 51.63 | 4.69 | 0.39 | 0.757 |
| Mid 00 | AA | FSTR 3 | 51.14 | 4.69 | −0.10 | 0.933 |
| Mid 00 | AA | FSTR 4 | 50.64 | 4.69 | −0.60 | 0.630 |
| Mid 00 | BB | FSTR 1 | 42.99 | 4.70 | −8.25 | 2.05E−07 |
| Mid 00 | BB | FSTR 2 | 44.28 | 4.71 | −6.96 | 1.36E−05 |
| Mid 00 | BB | FSTR 5 | 44.19 | 4.67 | −7.05 | 8.18E−12 |
| Mid 00 | BB | FSTR 3 | 44.48 | 4.68 | −6.76 | 6.69E−06 |
| Mid 00 | BB | FSTR 4 | 44.03 | 4.68 | −7.21 | 1.93E−06 |
| Mid 00 | CC | FSTR 1 | 44.95 | 4.69 | −6.29 | 2.68E−05 |
| Mid 00 | CC | FSTR 5 | 46.33 | 4.68 | −4.91 | 7.53E−07 |
| Mid 00 | CC | FSTR 3 | 45.95 | 4.71 | −5.29 | 0.00070 |
| Mid 00 | CC | FSTR 4 | 45.93 | 4.68 | −5.31 | 0.00035 |
| Mid 00 | DD | FSTR 1 | 51.32 | 4.69 | 0.08 | 0.955 |
| Mid 00 | DD | FSTR 2 | 53.09 | 4.71 | 1.85 | 0.234 |
| Mid 00 | DD | FSTR 5 | 53.09 | 4.67 | 1.85 | 0.049 |
| Mid 00 | DD | FSTR 3 | 52.46 | 4.68 | 1.23 | 0.398 |
| Mid 00 | DD | FSTR 4 | 52.85 | 4.68 | 1.61 | 0.264 |
| Mid 00 | EE | FSTR 1 | 51.56 | 4.69 | 0.32 | 0.829 |
| Mid 00 | EE | FSTR 2 | 53.04 | 4.69 | 1.80 | 0.229 |
| Mid 00 | EE | FSTR 5 | 53.18 | 4.68 | 1.94 | 0.042 |
| Mid 00 | EE | FSTR 3 | 52.85 | 4.68 | 1.61 | 0.269 |
| Mid 00 | EE | FSTR 4 | 52.71 | 4.69 | 1.47 | 0.320 |
| Mid 00 | FF | FSTR 1 | 50.28 | 4.69 | −0.96 | 0.511 |
| Mid 00 | FF | FSTR 2 | 51.55 | 4.70 | 0.31 | 0.836 |
| Mid 00 | FF | FSTR 5 | 51.41 | 4.67 | 0.17 | 0.854 |
| Mid 00 | FF | FSTR 3 | 51.47 | 4.69 | 0.23 | 0.874 |
| Mid 00 | FF | FSTR 4 | 51.08 | 4.69 | −0.16 | 0.911 |
| Mid 00 | CONTROL | CONTROL | 51.24 | 4.68 | 0.00 | |

(B)

| | | | | | | |
|---|---|---|---|---|---|---|
| Early 20 | GG | FSTR 1 | 60.82 | 4.51 | 3.85 | 0.0152 |
| Early 20 | GG | FSTR 2 | 60.48 | 4.51 | 3.51 | 0.0235 |
| Early 20 | GG | FSTR 5 | 59.28 | 4.51 | 2.32 | 0.0862 |
| Early 20 | GG | FSTR 3 | 60.69 | 4.55 | 3.73 | 0.0286 |
| Early 20 | GG | FSTR 4 | 60.07 | 4.54 | 3.10 | 0.0552 |
| Early 20 | HH | FSTR 1 | 58.55 | 4.52 | 1.59 | 0.0823 |
| Early 20 | HH | FSTR 2 | 58.19 | 4.51 | 1.23 | 0.1498 |
| Early 20 | HH | FSTR 3 | 58.46 | 4.56 | 1.49 | 0.1720 |
| Early 20 | HH | FSTR 4 | 57.67 | 4.54 | 0.70 | 0.4816 |
| Early 20 | II | FSTR 1 | 61.55 | 4.52 | 4.59 | 0.0041 |
| Early 20 | II | FSTR 2 | 61.53 | 4.51 | 4.57 | 0.0033 |
| Early 20 | II | FSTR 5 | 60.06 | 4.51 | 3.09 | 0.0218 |
| Early 20 | II | FSTR 3 | 61.48 | 4.55 | 4.51 | 0.0076 |
| Early 20 | II | FSTR 4 | 60.83 | 4.54 | 3.87 | 0.0175 |
| Early 20 | JJ | FSTR 1 | 63.33 | 4.52 | 6.37 | 8.89E−05 |
| Early 20 | JJ | FSTR 2 | 63.02 | 4.51 | 6.06 | 0.0002 |
| Early 20 | JJ | FSTR 5 | 61.68 | 4.51 | 4.72 | 0.0007 |
| Early 20 | JJ | FSTR 3 | 63.17 | 4.56 | 6.20 | 0.0004 |
| Early 20 | JJ | FSTR 4 | 62.24 | 4.53 | 5.28 | 0.0013 |
| Early 20 | KK | FSTR 1 | 62.61 | 4.52 | 5.65 | 0.0005 |
| Early 20 | KK | FSTR 2 | 62.18 | 4.51 | 5.21 | 0.0010 |
| Early 20 | KK | FSTR 5 | 61.04 | 4.51 | 4.08 | 0.0030 |
| Early 20 | KK | FSTR 3 | 62.34 | 4.55 | 5.38 | 0.0016 |
| Early 20 | KK | FSTR 4 | 61.87 | 4.53 | 4.91 | 0.0028 |
| Early 20 | LL | FSTR 1 | 63.70 | 4.51 | 6.73 | 3.28E−05 |
| Early 20 | LL | FSTR 2 | 63.63 | 4.51 | 6.67 | 2.89E−05 |
| Early 20 | LL | FSTR 5 | 62.03 | 4.51 | 5.06 | 0.0003 |
| Early 20 | LL | FSTR 3 | 63.71 | 4.56 | 6.75 | 0.0001 |
| Early 20 | LL | FSTR 4 | 62.95 | 4.54 | 5.98 | 0.0003 |
| Early 20 | HH | FSTR 5 | 56.96 | 4.51 | 0.00 | |
| | CONTROL | CONTROL | | | | |

(C)

| | | | | | | |
|---|---|---|---|---|---|---|
| Late 20 | MM | FSTR 1 | 59.16 | 1.47 | −10.25 | 1.54E−05 |
| Late 20 | MM | FSTR 2 | 60.81 | 1.47 | −8.60 | 0.0002 |
| Late 20 | MM | FSTR 5 | 60.56 | 1.47 | −8.85 | 4.32E−06 |
| Late 20 | NN | FSTR 1 | 60.71 | 1.47 | −8.70 | 0.0002 |
| Late 20 | NN | FSTR 2 | 62.36 | 1.47 | −7.05 | 0.0021 |
| Late 20 | NN | FSTR 5 | 62.11 | 1.47 | −7.30 | 0.0001 |
| Late 20 | OO | FSTR 1 | 63.02 | 1.47 | −6.39 | 0.0050 |
| Late 20 | OO | FSTR 2 | 64.67 | 1.47 | −4.74 | 0.0351 |
| Late 20 | OO | FSTR 5 | 64.42 | 1.47 | −4.99 | 0.0064 |
| Late 20 | PP | FSTR 1 | 68.01 | 1.47 | −1.40 | 0.2881 |
| Late 20 | PP | FSTR 2 | 69.66 | 1.47 | 0.25 | 0.8496 |
| Late 20 | A | FSTR 1 | 68.73 | 1.47 | −0.68 | 0.7599 |
| Late 20 | A | FSTR 2 | 70.38 | 1.47 | 0.97 | 0.6602 |
| Late 20 | A | FSTR 5 | 70.13 | 1.47 | 0.72 | 0.6844 |
| Late 20 | C | FSTR 1 | 68.34 | 1.47 | −1.07 | 0.6288 |
| Late 20 | C | FSTR 2 | 69.99 | 1.47 | 0.58 | 0.7935 |
| Late 20 | C | FSTR 5 | 69.74 | 1.47 | 0.33 | 0.8529 |
| Late 20 | PP | FSTR 5 | 69.41 | 1.47 | 0.00 | |
| | CONTROL | CONTROL | | | | |

Table 6 shows disease tolerance response, in presence and absence of a fungicide seed treatment, separated by relative field tolerance and relative maturity grouping for several soybean varieties. The table shows means for each treatment (variety×seed treatment combination), with the asterisk (*) indicating a statistically significant difference at p<0.1.

TABLE 6
Effect of seed treatments on Phytophthora tolerance by various
soybean varieties.

| | | | Test results | | |
|---|---|---|---|---|---|
| | | | | Mean | Mean |
| | | | Mean | disease | disease |
| | | | disease | tolerance | tolerance |
| | | Variety | tolerance | score | score |
| | | PRT | score | Fungicide | Fungicide |
| Soy- | | score | Untreated | seed | seed |
| bean | Relative | (pre- | Control | treatment 1* | treatment 2* |
| Variety | Maturity | assigned) | (scale 1-9) | (scale 1-9) | (scale 1-9) |
| 1 | 19 | 2 | 3.0 | 4.2 | 4.3 |
| 2 | 1 | 3 | 3.6 | 4.8 | 4.9 |
| 3 | 27 | 3 | 4.3 | 5.2 | 5.2 |
| 4 | 28 | 3 | 4.7 | 5.4 | 5.3 |
| 5 | 42 | 3 | 4.0 | 5.2 | 5.2 |
| 6 | 26 | 4 | 5.0 | 5.7 | 6.0 |
| 7 | 35 | 4 | 5.7 | 6.4 | 6.4 |
| 8 | 39 | 4 | 4.8 | 6.1 | 5.9 |
| 9 | −9 | 5 | 5.7 | 6.8 | 6.6 |
| 10 | 22 | 5 | 5.3 | 6.2 | 6.2 |
| 11 | 33 | 5 | 5.1 | 6.0 | 6.1 |
| 12 | 29 | 5 | 8.6 | 9.1 | 9.1 |
| 13 | 36 | 5 | 5.1 | 6.1 | 6.2 |
| 14 | 38 | 6 | 6.2 | 7.4 | 7.1 |
| 15 | 28 | 6 | 8.4 | 9.1 | 9.0 |
| 16 | 27 | 7 | 8.6 | 9.1 | 9.1 |
| 17 | 46 | 7 | 7.0 | 7.9 | 7.8 |
| 18 | 33 | 9 | 8.0 | 8.8 | 8.8 |

*p < 0.1 for all variety x seed treatment combinations, compared to untreated control.

The disease tolerance scores are relative and assigned from a numerical scale 1-9. Fungicide seed treatments 1 and 2 differ in the presence or rate (dosage) of one or more fungicides. The pre-assigned PRT score is the Phytopthora tolerance (PRT) score that was previously assigned to that variety as part of breeding performance trials. Mean disease tolerance fungicide seed treatment 1 indicates the assessed Phytopthora tolerance scores assigned to this variety based on visual assessment for the instant green house trial performed in Table 6 and the same applies for fungicide seed treatment 2. The untreated control did not have any fungicide seed treatment.

The data provided in Table 6 indicates that certain soybean varieties with a lower pre-assigned PRT score exhibited higher numerical increase in PRT scores in the presence of seed treatments compared to other soybean varieties with higher pre-assigned PRT scores. Nevertheless, even those soybean varieties with higher pre-assigned PRT scores demonstrated an increase in Phytophothora tolerance in the presence of seed treatments, thereby indicating benefits of including different modes of resistance to increasing disease tolerance.

The trial data further validates the total seed solution concept: (i) by including appropriate seed treatments earlier in the breeding program; (ii) diversifies the available germplasm with higher yield potential, albeit lacking higher disease tolerance; and (iii) to advance those varieties in the breeding pipeline and offer enhanced genetic diversity for growers.

### Example 2

### Corn Breeding Methods and Seed Treatment

A single cross maize hybrid results from the cross of two inbred varieties, each of which has a genotype that complements the genotype of the other. A hybrid progeny of the first generation is designated F1. In the development of commercial hybrids in a maize plant breeding program, only the F1 hybrid plants are sought. F1 hybrids are more vigorous than their inbred parents. This hybrid vigor, or heterosis, can be manifested in many polygenic traits, including increased vegetative growth and increased yield.

However, during the development of inbred parents or during the selection of inbred parents for breeding purposes, provision of one or more seed applied component to specifically target a trait deficiency of the inbred parents is desirable. Alternatively, the seed applied component is used to select inbred parents based on the performance of the F1 hybrids in the presence of the seed applied component.

One such embodiment is the method of crossing a maize variety with another maize plant, such as a different maize variety, to form a first generation F1 hybrid seed. The performance of the first generation F1 hybrid seed is evaluated in the presence of a seed applied component that was specifically selected to address a particular pest pressure (e.g., corn root worm) compared to the same F1 hybrid seeds grown in the absence of that seed applied component. The performance of the F1 hybrid plants based on the presence of the seed applied component is chosen and the corresponding parental inbreds are selected for further breeding. The first generation F1 hybrid seed, plant and plant part produced by this method is an embodiment. The first generation F1 seed, plant and plant part will comprise an essentially complete set of the alleles of a desirable variety. One of ordinary skill in the art can utilize molecular methods to identify a particular F1 hybrid plant produced. Further, one of ordinary skill in the art may also produce F1 hybrids with transgenic, male sterile and/or locus conversions.

For example, one or more seed applied component is selected to control a maize disease such as, Anthracnose, Bacterial Stalk Rot, Common Rust, Fusarium Stalk Rot, Fusarium Root Rot, Gray Leaf Spot, Maize Chlorotic Mottle Virus, Southern Rust, Stewart's Wilt, Common Smut, Goss's Wilt, Head Smut, Nematodes, and Physoderma.

In an embodiment, one or more inbred parents are selected based on their ability to produce F1 hybrids, whose performance in the presence of the seed treatment is evaluated as a whole to determine the suitability of that F1 hybrid for a particular geographical location or for a location with a particular disease or pest pressure. The development of a maize hybrid in a maize plant breeding program involves three steps: (1) the selection of plants from various germplasm pools for initial breeding crosses; (2) the selfing of the selected plants from the breeding crosses for several generations to produce a series of varieties, although different from each other, breed true and are highly uniform; and (3) crossing the selected varieties with different varieties to produce the hybrids. Within the traditional breeding program, seed applied component is now used as a factor in selecting the F1 hybrid performance and adapting inbreds selected for their performance in a certain geographical zone to a different geographical zone or a zone that contains a different pest pressure, disease pressure, soil type, climate and other environmental factors with complementation provided by one or more seed applied components.

Applications involving the use of seed applied components earlier in the breeding processes are also used to produce a single cross hybrid, a double cross hybrid, or a three-way hybrid. A single cross hybrid is produced when two inbred varieties are crossed to produce the F1 progeny. A double cross hybrid is produced from four inbred varieties crossed in pairs (A×B and C×D) and then the two F1 hybrids are crossed again (A×B)×(C×D). A three-way cross hybrid is produced from three inbred varieties where two of the inbred varieties are crossed (A×B) and then the resulting F1 hybrid is crossed with the third inbred (A×B×C).

For example, quality emergence of corn seedlings in stressed conditions (e.g., cool, moist soils) is an important consideration for corn seed production and breeding programs. Accordingly, selection of parent inbreds and resulting hybrids, accounts for emergence characteristics. Seed treatment technologies aid in emergence, seedling vigor, stand count, and other early season plant characteristics, which impact yield. For example, a particular corn inbred that does not have adequate emergence or resistance to an early season seedling disease is chosen or selected such that with application of a seed treatment, that particular inbred line becomes a suitable parental material for further breeding, or in the case of a hybrid, for commercial advancement and placement of that hybrid in a high disease pressure or stress location with the presence of a suitable seed treatment. Further, based on precision agriculture, a specific location is supplied with a specific variety or hybrid treated with a specific seed treatment at a particular dose.

In a limited field trial, experiments were performed with 6 representative commercial corn hybrids to 5 different seed treatments under different emergence conditions that included stressed conditions such as cool and moist soil. Stand count and the resultant yield were measured following treatment with the 5 different seed treatment recipes, one of which was considered as a “control”. No statistically significant variation in yield were found for the seed treatment by hybrid under the limited number of locations and conditions tested, but numerical trends indicated potential small differences. Statistically significant differences were observed among the hybrids tested for stand count with respect to the tested seed treatments. Small differences for hybrids are indicative of the potential for larger differences when inbreds are tested. Experiments are performed at the earlier stage inbred level to evaluate the impact of seed treatments on selecting parental lines for hybrid production.

### Example 3

### Improving Canola Yield Through Breeding and Seed Treatment

Similar to soybean breeding methods described herein, canola breeding programs utilize techniques such as mass and recurrent selection, backcrossing, pedigree breeding and haploidy. For a general description of rapeseed and Canola breeding, see, Downey and Rakow, (1987) “Rapeseed and Mustard” In: Principles of Cultivar Development, Fehr, (ed.), pp 437-486; New York; Macmillan and Co.; Thompson, (1983) “Breeding winter oilseed rape Brassica napus”; Advances in Applied Biology 7:1-104; and Ward, et. al., (1985) Oilseed Rape, Farming Press Ltd., Wharfedale Road, Ipswich, Suffolk, each of which is hereby incorporated by reference.

Canola breeding utilizes a pollination control system for effective transfer of pollen from one parent to the other and an effective method for producing hybrid canola seed and plants. For example, the Ogura cytoplasmic male sterility (CMS) system, developed via protoplast fusion between radish (Raphanus sativus) and rapeseed (Brassica napus), is one of the most frequently used methods of hybrid production for canola. The traditional breeding methods for canola are improved by applying one or more seed applied components to earlier stages in the breeding process. For example, for most traits the true genotypic value may be masked by other confounding plant traits or environmental factors. Seed treatment or seed applied component, when used earlier in the breeding process unlocks that genetic value for developing varieties or hybrids of canola. One method for identifying a superior plant is to observe its performance relative to other experimental plants and to one or more widely grown standard varieties in the presence of one or more seed treatments.

To select and develop a superior hybrid, it is necessary to identify and select genetically unique individuals that occur in a segregating population. With the help of seed applied component, such segregating populations are better screened to identify individuals that are selected to perform in a chosen environment. The segregating population is the result of a combination of crossover events plus the independent assortment of specific combinations of alleles at many gene loci that results in specific and unique genotypes in the presence of a particular seed applied component such as an insecticide. Once such a variety is developed its value to society is substantial since it is important to advance the germplasm base as a whole in order to maintain or improve traits such as yield, disease resistance, pest resistance and plant performance in extreme weather conditions. Locus conversions are routinely used to add or modify one or a few traits of such a line and this further enhances its value and usefulness to society.

Backcrossing can be used to improve inbred varieties and a hybrid variety which is made using those inbreds. Backcrossing can be used to transfer a specific desirable trait from one variety, the donor parent, to an inbred called the recurrent parent which has overall good agronomic characteristics yet that lacks the desirable trait. Backcrossing is done in the presence of a particular seed treatment used to overcome a deficiency or to further enhance a quality of the population. This transfer of the desirable trait into an inbred with overall good agronomic characteristics can be accomplished by first crossing a recurrent parent to a donor parent (non-recurrent parent). The progeny of this cross is then mated back to the recurrent parent followed by selection in the resultant progeny for the desired trait to be transferred from the non-recurrent parent. These backcrossings are done in the presence or absence of one or more seed treatments.

Molecular markers can also be used during the breeding process for the selection of qualitative traits in addition to seed treatments to accompany particular varieties. For example, markers can be used to select plants that contain the alleles of interest during a backcrossing breeding program. The markers can also be used to select for the genome of the recurrent parent and against the genome of the donor parent. Using this procedure can minimize the amount of genome from the donor parent that remains in the selected plants and perform better with one or more seed treatments.

Clubroot (Plasmodiophora brassicae) is a devasting disease that infects cruciferous crops, e.g., canola (or oilseed rape). Accordingly, it is a significant target for canola or oilseed rape breeding programs. A seed-applied technology that provides clubroot protection complements canola germplasm with moderate resistance, thus increasing the availability of canola germplasm pool of varying disease resistance potential for additional breeding and selection that otherwise would not have been selected.

In addition to soy, corn and canola, other crop breeding programs that could benefit from utilization of seed-applied technologies as a tool of the breeding program include rice, sorghum, wheat and sunflower. For example, downy mildew (Plasmopara halstedii) is a significant disease of sunflower. Downy mildew results in stunted plants early in the growth cycle and the plants often wither and die. The infected plants may also continue to develop with an erect and horizontal head with little or no seed. The disease symptoms on seedlings include yellowing of the leaves along with a white fungal growth on the lower leaf surfaces. Secondary infection is also possible in the four to eight leaf stages. Downy mildew resistance and tolerance is therefore a target for sunflower breeding programs; for example, resistance to certain races of downy mildew have been found in breeding lines, introductions and specifically, resistance in certain lines has been found to be due to a single dominant gene, designated PI genes. Utilizing fungicide seed treatments targeted for downy mildew, such as acibenzolar or oxathiapiprolin, alongside host resistance developed from a breeding program can allow for selection of higher performing sunflower lines.

For example certain sunflower varieties having certain resistance genes and other varieties with different resistance genes may perform differently with seed applied components, including fungicide seed treatment. For example, a sunflower variety (with seed treatment) but without a resistance gene outyields a another sunflower variety with the resistance gene but without seed treatment. Therefore, seed treatment as a breeding tool to breed high yield varieties even in the presence of pest or disease pressure and in the absence of all necessary native resistance genes, is a valuable option for sunflower breeding.

### Example 4

### Alteration of Plant Maturity by Seed Applied Component

In an embodiment, varieties of soybean seeds are treated with a seed applied component, such as for example, a fungicide and/or an insecticide and planted in a crop growing environment. Relative maturity of plants such as soybeans can be changed by seed treatment applications, thus enabling breeding or increasing seed yield to suit a particular growing environment, where the particular soybean variety does not belong to the appropriate maturity group. For example, soybean varieties having a lower maturity group can be treated with a suitable seed applied component and planted in a geographic region suitable for a soybean variety that belong to a higher maturity group.

Soybean varieties are generally divided into groups according to their relative times of maturity. These maturity groups (MGs) are usually designated by Roman numerals, from 0 (or multiple zeroes, for very short-season varieties) to maturity group IX or higher for types developed for warmer climates with shorter days during the growing season. A decimal to the MG number was also added, for example, to a variety as MG 3.2 or 4.6, to indicate finer gradations within a single maturity group or between two maturity groups. Further, maturity group ratings for soybean can also be based on a numerical scale that is different than the Roman numerals. For example, as described in Tables 2 and 3, the maturity group is based on a relative maturity scale that includes, e.g., mid 30, late 40, late 30 and so on and so forth. Nevertheless, one of ordinary skill in the art would know what a maturity rating and group are for soybeans and other crops such as wheat, rice, sorghum, canola as applicable.

For example, varieties of MG I can be grown in northern midwestern states such as Minnesota; however they are not adapted to grow and produce high yields in warmer Southern states like Southern Indiana and Arkansas. In another example, varieties of MG IV are best adapted to grown in southern Indiana.

Growing soybeans that are adapted to effectively use the full growing season in a particular crop growing environment is highly beneficial to yield. For example, if a particular soybean variety is able to survive early season frost and still able to germinate and set seed before the growing is season is complete, the soybean variety is positioned to maximize yield. However, a late maturity soybean variety may not be able to survive the early season cold conditions that often prevail in northern climatic conditions. However, with a seed applied component that improves the survivability of the germinating seed and the early seedling in those conditions, a late season maturity soybean variety can be planted in northern climatic zones.

Alternatively, in some cases, early maturity is desired to avoid the hot and dry conditions that often prevail in the Southern states of the US. In those circumstances, a seed treatment is useful to change the late season maturity varieties to early maturity hybrids, by for example, promoting earlier germination and growth compared to seeds not treated with that seed treatment.

Further, seed treatments can also help reduce lodging in high yielding environment and later in the season, thereby maximizing yield for those varieties that mature early or later in the growing cycle.

Similar to soybeans, maturity is also altered for corn, wheat, rice, sorghum and canola where such alteration of maturity is suitable to increase yield in crop growing environments for such crops. The dosage of the seed treatments and the planting window can vary depending on the crop, soil conditions, geographical area and disease pressure present in that area or location.

Heat units (HU) are used to explain temperature impact on rate of corn development, and these HUs provide growers an indexing system for selection of corn hybrids in a given location. Several formulas exist for the calculation of heat units. Among them, GDD or GDU (Growing Degree Day or Growing Degree Unit) and CHU (Crop Heat Units) are most commonly used. GTI (General Thermal Index) has recently been developed that attempts to improve accuracy in predicting developmental stages.

GDDs, also known as GDUs, are often referred to simply as HUs in the US. The method to calculate GDD is to average daily temperature (degrees F.) then minus 50, proposed by the National Oceanic and Atmospheric Administration and labeled as the “Modified Growing Degree Day”.

GDU=(Tₘₐₓ+Tₘᵢₙ)/2−Tbase

Where Tₘₐₓ is maximum daily temperature, Tₘᵢₙ is minimum daily temperature, and Tbase is a base temperature (mostly set at 50 F).

CHUs are first developed and used in Ontario, Canada in the 1960's. The method to calculate CHU is somewhat more complex, allocating different responses of development to temperature (degrees C.) between the day and the night.

CHU_{day}=3.33*(Tₘₐₓ−10)−0.084*(Tₘₐₓ−10)2

CHU_{night}=1.8*(Tₘᵢₙ−4.4)

CHU=[CHU_{day}+CHU_{night}]/2

GTIs are calculated based on different responses of corn from planting to silking and from silking to maturity. The period between planting and silking is defined as vegetative growth, whereas time from silking to maturity is the grain filling stage.

F_{T(veg)}=0.0432T²−0.000894T³

F_{T(fill)}=5.358+0.011178T²

GTI=F_{T(veg)}+F_{T(fill)}

Where T is mean daily temperature (degrees C.), F_{T(veg)} is for the period from planting to silking, F_{T(fill)} is for the period from silking to maturity.

Relative Maturity Conversion Guidelines

Guidelines for converting various relative-maturity rating systems have been reported by Dwyer, et al., (Agron. J. 91:946-949). Conversions for CHU, GDD and the Corn Relative Maturity rating system (CRM), also referred to as the Minnesota Relative Maturity Rating, are generally available. The CRM rating system is widely used in the US to characterize hybrid relative maturity. The CRM rating is not based on temperature, but on the duration in days from planting to maturity (in an average year) relative to a set of standard hybrids. The approximate conversion from one rating system to another can be estimated from a linear regression equation. Some data sets calculate GDDs from degree Fahrenheit, resulting in a number that is 1.8× larger than that when using degree Celcius in the estimation of CHU or CRM from GDD (or 1.8× smaller when estimating GGD from CHU or CRM). (University of Guelph Publication; Corn Maturity and Heat Units, can be accessed via plant.uoguelph.ca/research/homepages/ttollena/research/cropheatunits.html, using the prefix www).

Maturity may also generally refer to a physiological state, where maximum weight per kernel has been achieved for the planted corn. This is often referred to as physiological maturity and is generally associated with the formation of an abscission layer or “black layer” at the base of the kernel. One of the most commonly used methods for designating hybrid maturity ratings (days to maturity) is based on comparisons among hybrids close to the time of harvest.

Seed treatment is used to alter the CRM values for corn inbred and hybrids by for example, about 5-15 CRM or 5-7 CRM, 7-10 CRM or 10-15 CRM. In an embodiment, parent lines (inbreds) selected for breeding or hybrid production may not have the same maturity or CRM ranges. However, a particular seed treatment can be applied to one or both the parents such that the both the parental lines reach appropriate and relevant physiological maturity (e.g., during flowering/pollination and silking) to maximize pollination and seed set.

What is claimed is:

1. A method of increasing crop yield under pest or disease pressure, the method comprising:
a. providing a first crop plant that is adapted to grow in a first crop growing environment in the absence of a substantial pest or disease pressure and wherein the first crop plant does not exhibit substantial resistance to one or more pests or diseases;
b. providing a second crop plant that is adapted to grow in a second crop growing environment in the presence of a substantial pest or disease pressure and wherein the second crop plant exhibits substantial resistance to one or more pests or disease but yields less compared to the first crop plant in the absence of said pest or diseases pressure;
c. crossing the first and the second crop plant;
d. obtaining a plurality of seeds from the cross;
e. growing the plurality of seeds treated with one or more seed treatments that enhance resistance to one or more pests or diseases present in the second crop growing environment;
f. increasing crop yield by selecting for one or more progeny plants that yield higher under the presence of the substantial pest or disease pressure in the second crop growing environment.

2. (canceled)

3. The method of claim 1, wherein the first and second crop plants are selected from the group consisting of soybean, maize, rice, wheat and canola.

4. The method of claim 1, wherein the disease is soybean sudden death syndrome.

5. The method of claim 1, wherein the pest is selected from the group consisting of wireworm, white grubs, black cutworms, seedcorn maggot, corn root worm, fall armyworm, flea beetle and cutworms.

6. A method of developing an integrated seed product comprising a seed applied component, the method comprising:
a. growing a population of seeds treated with a seed applied component, wherein the population of seeds exhibit genetic variability with respect to one or more agronomic traits and wherein the seed applied component improves the one or more agronomic traits;
b. selecting one or more plants that exhibit increased agronomic performance in the presence of the seed applied component for further breeding; and
c. developing an integrated seed product comprising the seed applied component, wherein the seed applied component enhances the performance of the one or more agronomic traits.

7. (canceled)

8. (canceled)

9. (canceled)

10. The method of claim 6, wherein the seed applied component on the plurality of seeds for the parental population increases an agronomic characteristic selected from the group consisting of seed germination, yield, plant health, disease, pest resistance, and a combination thereof.

11. The method of claim 6, wherein the seed applied component to the plurality of seeds for the parental population improves an agronomic characteristic selected from the group consisting of seed germination, yield, plant health, disease, pest resistance, and a combination thereof, for the progeny plants.

12. (canceled)

13. A method of reducing the development of a resistant insect through an integrated refuge comprising:
(a) providing a first portion of seeds coated with a first seed applied insecticide, and
(b) providing a second portion of seeds coated with a second seed applied insecticide,
wherein the first and second seed applied insecticides function through distinct modes of action in controlling one or more insects, and wherein the first and second portions of seeds are present in the same container for planting in a field.

14. The method of claim 13, wherein the first portion of the seeds contain a trait not present in the second portion of the seeds.

15. The method of claim 13, wherein the seeds are maize seeds.

16. The method of claim 13, wherein the second portion of seeds comprise about 5% to about 25% of the total number of seeds in the container.

17. The method of claim 14, wherein the trait is a transgenic trait.

18. The method of claim 17, wherein the transgenic trait is due to the expression of a Bacillus thuringiensis insecticidal protein, a bacterial insecticidal protein, a plant insecticidal protein, a RNA targeting one or more insects, or a combination thereof.

19. A method of developing a specific population of crop seeds coated with a specific seed applied component for a specific location, the method comprising
(a) providing the specific population of crop seeds coated with the specific seed applied component, wherein the specific population of seeds is selected to exhibit a desirable characteristic in the presence of the specific seed applied component and wherein the seed applied component is provided for its performance against one or more pests present at the specific location, and
(b) growing the specific population of crop seeds in a crop growing environment in the specific location.

20. The method of claim 19, wherein the specific population of crop seeds exhibit disease tolerance or insect resistance.

21. The method of claim 19, wherein the specific population of crop seeds exhibit enhanced plant vigor.

22. The method of claim 19, wherein the seed applied component is applied at a rate lower than the rate normally used for a general population of crop seeds and wherein the yield resulting from the specific population of crop seeds is the same or higher as compared to the general population of crop seeds.

23. The method of claim 22, wherein the specific population of crop seeds is soybean.

24. The method of claim 22, wherein the specific population of crop seeds is selected from the group consisting of soybean, maize, rice, sorghum, alfalfa, canola, cotton and wheat.

25. The method of claim 19, wherein the specific location is chosen based on an environmental factor selected from the group consisting of pest pressure, disease pressure, soil type, temperature, humidity, day length and a combination thereof.

26-32. (canceled)
